(19) United States
(12) Patent Application Publication
(10) Pub. No.: US 2010/0129792 A1
(43) Pub. Date: May 27, 2010

(54) DIRECT MONITORING AND PCR AMPLIFICATION OF THE DOSAGE AND DOSAGE DIFFERENCE BETWEEN TARGET GENETIC REGIONS

(76) Inventor: Gerassimos Makrigiorgos, Chestnut Hill, MA (US)

Correspondence Address: FOLEY & LARDNER LLP, 150 EAST GILMAN STREET, P.O. BOX 1497, MADISON, WI, 53701-1497, US

(21) Appl. No.: 11/965,971

(22) Filed: Dec. 28, 2007

Related U.S. Application Data
(60) Provisional application No. 60/966,532, filed on Feb. 6, 2007.

(51) Int. Cl. C12Q 1/68

(52) U.S. Cl. 435/6

(57) ABSTRACT

Disclosed herein are methods of detecting target nucleic acids. In particular, methods for anti-primer quenching real-time PCR (aQRT-PCR) are described. The methods provide for detection of target nucleic acids in simplex or multiplex formats for gene copy number determination and SNP-genotyping. Also described are methods for determining the dosage difference between two target nucleic acids.

### CROSS-REFERENCE TO RELATED APPLICATIONS

This application claims priority to U.S. Patent Application No. 60/966,532, filed Feb. 6, 2007.

### FIELD OF THE INVENTION

The present invention relates generally to the field of detecting nucleic acids. In particular, the present invention relates to nucleic acid amplification detection methods using an interactive primer and anti-primer.

### BACKGROUND OF THE INVENTION

The following description is provided to assist the understanding of the reader. None of the information provided or references cited is admitted to be prior art to the present invention.

Amplification of target nucleic acids continues to be important for genetic diagnostics and drug discovery. Amplification by polymerase chain reaction (PCR) is based on repeated cycles of the following steps: denaturation of double-stranded DNA followed by oligonucleotide primer annealing to the DNA template, and primer extension by a nucleic acid polymerase. The oligonucleotide primers used in PCR are designed to anneal to opposite strands of the DNA, and are positioned so that the nucleic acid polymerase-catalyzed extension product of one primer can serve as the template strand for the other primer. The PCR amplification process results in the exponential increase of discrete DNA fragments whose length is defined by the 5′ ends of the oligonucleotide primers. A major advance for PCR-based nucleic acid detection, quantification and genotyping has been the development of homogenous, closed-tube assays using fluorescence detection that facilitate high-throughput detection and minimize the likelihood of false-positive results owing to carryover contamination.

In some instances, oligonucleotide sequences differ by only a few nucleotides, as in the case of many allelic sequences. Single nucleotide polymorphisms (SNPs) refer to alleles that differ by a single nucleotide. Even this single nucleotide difference can, at least in some instances, change the associated genetic response or traits. Accordingly, to determine which allele is present in a sample, an assay technique must be sensitive to distinguish between closely related sequences.

For example, amplification of oncogenes such as Her-2 is crucial in the development of certain forms of breast cancer (Lin, Broadwater et al. 2004) while amplification of EGFR gene is associated with lung cancer (Paez, Janne et al. 2004). Similarly, tumor development often proceeds through inactivation of tumor-suppressor genes (Kinzler and Vogelstein 1996), thus if one of two copies of a tumor suppressor gene is inactivated due to a mutation (heterozygosity), subsequent loss of the second copy may allow the tumor formation process to proceed. Such allelic imbalances are considered a hallmark of cancer and a useful marker to detect in clinical samples (Kallioniemi et al. 1994).

Another type of biallelic inactivation in cancer occurs via combination of genetic and epigenetic modifications. For example, expression of one allele of a gene can be inhibited via promoter methylation while the second allele can be lost due to deletion, mutation or methylation (Jirtle, Sander et al. 2000). Finally, loss of imprinting is another emerging mechanism for epigenetic changes associated with cancer (Jirtle, Sander et al. 2000). Accurate and sensitive detection of such genetic and epigenetic changes between genomic regions in surgical specimens, biopsies or bodily fluids is of paramount importance for early cancer detection and prognosis of therapeutic efficacy, as well as for identification of cancer biomarkers.

### SUMMARY OF THE INVENTION

The present invention relates to the detection of nucleic acids using anti-primer quenching. In one aspect, the invention provides a method comprising: (a) contacting a sample to be tested for the presence of a target nucleic acid with (i) a primer comprising a first label and a first and a second region of nucleotides, wherein the first region of nucleotides is complementary to the target nucleic acid; and (ii) an anti-primer comprising a second label and a nucleotide sequence complementary to the second region of the primer, wherein the second label is capable of quenching a detectable signal from the first label, and further wherein the sample is contacted under conditions wherein the primer specifically hybridizes to the target nucleic acid, if present in the sample; (b) performing an amplification reaction with the primer to produce an amplification product having an incorporated primer; and (c) detecting the presence of the target nucleic acid in the sample by detecting the first label of the incorporated primer under conditions wherein the anti-primer specifically hybridizes to the second region of the primer and hybridization between the primer and the anti-primer quenches the detectable signal from unincorporated primer. In a particular embodiment the second region of the primer has a sequence according to SEQ ID NO: 2 and the anti-primer has a sequence according to SEQ ID NO: 1.

In one embodiment, the first and second label comprise a fluorophore/quencher pair. For example, the first label may be a fluorophore and the second label may be a quencher. In some embodiments, the fluorophore is selected from the group consisting of: FAM, TAMRA, ROX, Cy5, Cy3, and BODIPY, and the quencher is a dark quencher. In a suitable embodiment, the fluorophore is FAM or ROX and the quencher is a black hole quencher, BHQ™.

In one embodiment, the melting temperature of the first region of the first primer is higher than the melting temperature of the anti-primer. For example, the melting temperature of the first region of the first primer is from 5 to 10 degrees Celsius higher than the melting temperature of the anti-primer. Accordingly, the step of detecting the first label may comprise lowering the temperature of the reaction below the melting temperature of the anti-primer and measuring the signal from the first label.

In one embodiment, the methods may be performed in a multiplex format, i.e. to detect two or more target nucleic acids in a single reaction. Thus, the sample may be contacted with one or more additional primers, each primer comprising a label and a first and a second region of nucleotides, wherein the first region of nucleotides is complementary to additional target nucleic acids.

In a second aspect, the invention provides a method comprising: (a) contacting a sample to be tested for the relative amount of two target nucleic acids with (i) a first primer comprising a first label, a first and a second region of nucleotides and a non-extendible linker between the first and second region of nucleotides, wherein the first region of nucleotides is complementary to a first target nucleic acid; (ii) a second primer comprising a second label, a first and second region of nucleotides, and a non-extendible linker between the first and second region of nucleotides, wherein the first region of nucleotides is complementary to a second target nucleic acid and the second region of the second primer is complementary to the second region of the first primer and the second label is capable of quenching a detectable signal from the first label; (iii) a first anti-primer comprising a third label and a nucleotide sequence complementary to the first primer, wherein the third label is capable of quenching a detectable signal from the first label; and (iv) a second anti-primer comprising a fourth label and a nucleotide sequence complementary to the second primer, wherein the fourth label is capable of quenching a detectable signal from the second label, and further wherein the sample is contacted under conditions wherein the primers specifically hybridize to the target nucleic acids, if present in the sample; (b) performing an amplification reaction with the primers to produce amplification products having incorporated primers; (c) detecting the relative amount of the two target nucleic acids in the sample by detecting the first label and the second label of the incorporated primers under conditions where the anti-primers specifically hybridize to the primers and hybridization between the primer and the anti-primer quenches the detectable signal from unincorporated primer, and the second region of the first primer hybridizes to the second region of the second primer and hybridization between the second region of the first primer and the second region of the second primer quenches the detectable signal from the hybridized amplification products.

In one embodiment, the first label and second label each comprise a fluorophore, where the fluorophores may be the same or different. In certain embodiments, the first and second labels are independently selected from the group consisting of: FAM, TAMRA, ROX, CY5, CY3, and BODIPY, and the third and fourth labels each comprise a dark quencher. In a particular embodiment, the first and second labels are independently selected from the fluorophores FAM or ROX. For example, the quencher may be a dark quencher, e.g. a black hole quencher (BHQ).

The methods may also be used to measure the absolute amount of each of the target nucleic acids. In some embodiments, the amount of each of the target nucleic acids can be determined by increasing the temperature of the reaction to separate the hybridized second region of the first primer and the second region of the second primer and detecting the signal from one or both of the first label and the second label.

In some embodiments, the melting temperatures of the first regions of the first and second primers are both higher than the melting temperatures of the anti-primers. In a particular embodiment, the melting temperatures first regions of the first and second primers are from 5 to 10 degrees Celsius higher than the melting temperatures of the antiprimers.

The methods may be used discriminate between two related genetic sequences. For example, in one embodiment, the first and second target nucleic acids are alleles of a genetic locus. In a particular embodiment, the first and second target nucleic acids differ by a single nucleotide.

### BRIEF DESCRIPTION OF THE DRAWINGS

FIG. 1 is a schematic representation of the detection of a single target nucleic acid using anti-primer-based quenching. The anti-primer is used to directly quench the tail region of the free (unincorporated) primer when the temperature is lowered, while the incorporated primer generates a detectable signal.

FIG. 2A is a schematic representation of primers designed to detect two different target nucleic acids. FIG. 2B is a schematic representation of the interaction between the primers and the anti-primers.

FIG. 3 is a schematic representation of the primer/anti-primer pairs used in one embodiment where a dosage difference between two target nucleic acids is determined.

FIG. 4 is a schematic representation for the determination of a dosage difference between two genetic targets. In this embodiment, the fluorescence signal is directly proportional to the amount of dosage difference between genetic targets 1 and 2.

FIG. 5 presents a series of graphs comparing simplex aQRT-PCR with TaqMan®-based real-time PCR for the HER-2 gene. FIG. 5A shows the primary amplification curve for aQRT-PCR. FIG. 5B shows the log concentration of input genomic DNA vs. Ct for aQRT-PCR. FIG. 5C shows the primary amplification curve for the TaqMan® assay. FIG. 5D shows the log concentration of input genomic DNA vs. Ct for the TaqMan® assay.

FIG. 6 shows graphs of the real-time PCR quantification of four target nucleic acids: MYC (FIG. 6A), TOP1 (FIG. 6B), TBP (FIG. 6C), and HBEGF (FIG. 6D). One PCR reaction was performed for each target. Primary growth curves starting from serially diluted input genomic DNA (0.1-100 ng) are shown.

FIG. 7 shows the results of multiplex aQRT-PCR for the oncogene HER-2 and the housekeeping gene GAPDH.

FIG. 8 shows graphs of the detection of absolute dosage of two genetic targets in a multiplex real-time PCR reaction that determined the relative ratio of the HER-2 oncogene to the GAPDH housekeeping gene. In FIGS. 8A and 8B, GAPDH and HER-2 are detected in microdissected breast cancer samples, respectively. In FIGS. 8C and 8D, GAPDH and HER-2 are detected in formalin-fixed, paraffin embedded (FFPE) specimens. In FIGS. 8E and 8F, GAPDH and HER-2 are detected in plasma-circulating DNA from four blood samples.

FIG. 9 shows the detection of two genetic targets in a multiplex aQRT-PCR reaction. Simplex and multiplex aQRT PCR genotyping of the apolipoprotein B single nucleotide polymorphism B71 are depicted. FIGS. 9A and 9B show graphs of simplex allele-specific aQRT-PCR. FIGS. 9C and 9D shows multiplex aQRT-PCR genotyping on DNA predicted to be C/C, C/T, or T/T genotype. FIG. 9E shows the results of 10 independent repeats of the multiplex experiment for the 3 genomic DNAs.

### DETAILED DESCRIPTION

Disclosed herein are methods of detecting target nucleic acids. In particular, methods for anti-primer quenching real-time PCR (aQRT-PCR) are described. The methods provide for detection of target nucleic acids in simplex or multiplex formats for gene copy number determination and SNP-genotyping. The methods also provide for the determination of a dosage difference between two related genetic loci.

The present inventors have discovered a method for generating and detecting genetic targets using nucleic acid amplification. The methods may be used to detect single or multiple target nucleic acids. In particular, the methods are useful for genotyping, viral load testing, pathogen detection, and blood bank screening of infectious agents.

The methods overcome certain detection difficulties encountered by probe and primer-based approaches, and provide for both homogeneous (real time) and endpoint approaches in the absolute quantification of genetic targets of medical or biological relevance. Most real time PCR methods use the relief of fluorescence quenching as a way to generate the fluorescent signal during PCR. To achieve this, the quenching molecule has to be very effective in quenching the fluorescence of the fluorophore; otherwise, the increased signal over background during real time PCR is weak. For example, the probe-based TaqMan® approach uses an oligonucleotide labeled with a fluorophore and a quencher at the two ends in order to generate the signal during real time PCR. A detectable amount of background fluorescence is generated because the relatively large distance between the fluorophore and the quencher results in incomplete quenching. The present methods advantageously allow the quencher to be placed at any position along the anti-primer, e.g., in close proximity to the fluorophore. Therefore, background fluorescence is minimized. In a preferred embodiment, a fluorescent label is placed on the 5′ end of the primer and a quencher is placed on the 3′ end of the anti-primer. Thus, the distance between the label and the quencher is only a few Angstroms, thereby achieving almost 100% quenching. As a result, the present methods allow the flexibility to achieve ideal quenching of the fluorescent label, which results in strong signal generation over background during PCR.

Another advantage of the present methods is that only a single quencher-labeled anti-primer is required to quench labeled primers directed to many different target nucleic acids. In one embodiment, the primer(s) to which the anti-primer binds contain the same generic oligonucleotide tail. The result of requiring fewer oligonucleotides and a singly labeled anti-primer facilitates the design of the assays and reduces the overall cost relative to approaches that require double-labeling of each individual probe that is specific for each gene.

In the description that follows, a number of terms are utilized extensively. Definitions are herein provided to facilitate understanding of the invention. The terms defined below are more fully defined by reference to the specification as a whole.

Units, prefixes, and symbols may be denoted in their accepted SI form. Unless otherwise indicated, nucleic acids are written left to right in 5′ to 3′ orientation; amino acid sequences are written left to right in amino to carboxy orientation. Amino acids may be referred to herein by either their commonly known three letter symbols or by the one-letter symbols recommended by the IUPAC-IUBMB Nomenclature Commission. Nucleotides, likewise, may be referred to by their commonly accepted single-letter codes.

The terms “a” and “an” as used herein mean “one or more” unless the singular is expressly specified.

As used herein, “about” means plus or minus 10% unless otherwise indicated.

The terms “amplification” or “amplify” as used herein includes methods for copying a target nucleic acid, thereby increasing the number of copies of a selected nucleic acid sequence. Amplification may be exponential or linear. A target nucleic acid may be either DNA or RNA. The sequences amplified in this manner form an “amplicon” or “amplification product.” While the exemplary methods described hereinafter relate to amplification using the polymerase chain reaction (PCR), numerous other methods are known in the art for amplification of nucleic acids (e.g., isothermal methods, rolling circle methods, etc.). The skilled artisan will understand that these other methods may be used either in place of, or together with, PCR methods. See, e.g., Saiki, “Amplification of Genomic DNA” in PCR Protocols, Innis et al., Eds., Academic Press, San Diego, Calif. 1990, pp 13-20; Wharam, et al., Nucleic Acids Res. 2001 Jun. 1; 29(11):E54-E54; Hafner, et al., Biotechniques 2001 30(4):852-6, 858, 860; Zhong, et al., Biotechniques 2001 30(4):852-6, 858, 860.

The term “complement” “complementary” or “complementarity” as used herein with reference to polynucleotides (i.e., a sequence of nucleotides such as an oligonucleotide or a target nucleic acid) refers to standard Watson/Crick pairing rules. The complement of a nucleic acid sequence such that the 5′ end of one sequence is paired with the 3′ end of the other, is in “antiparallel association.” For example, the sequence “5′-A-G-T-3′” is complementary to the sequence “3′-T-C-A-5′.” Certain bases not commonly found in natural nucleic acids may be included in the nucleic acids described herein; these include, for example, inosine, 7-deazaguanine, Locked Nucleic Acids (LNA), and Peptide Nucleic Acids (PNA). Complementarity need not be perfect; stable duplexes may contain mismatched base pairs, degenerative, or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs. A complement sequence can also be a sequence of RNA complementary to the DNA sequence or its complement sequence, and can also be a cDNA. The term “substantially complementary” as used herein means that two sequences specifically hybridize (defined below). The skilled artisan will understand that substantially complementary sequences need not hybridize along their entire length.

A “fragment” in the context of a nucleic acid refers to a sequence of contiguous nucleotide residues which are at least about 5 nucleotides, at least about 7 nucleotides, at least about 9 nucleotides, at least about 11 nucleotides, or at least about 17 nucleotides. The fragment is typically less than about 300 nucleotides, less than about 100 nucleotides, less than about 75 nucleotides, less than about 50 nucleotides, or less than 30 nucleotides. Thus, fragments encompass a range of nucleotide sequences including combination of the listed lower and upper limits. In certain embodiments, the fragments can be used in polymerase chain reaction (PCR) or various hybridization procedures to identify or amplify identical or related parts of mRNA or DNA molecules. A fragment or segment may uniquely identify each polynucleotide sequence of the present invention.

“Genomic nucleic acid” or “genomic DNA” refers to some or all of the DNA from a chromosome. Genomic DNA may be intact or fragmented (e.g., digested with restriction endonucleases by methods known in the art). Methods of purifying DNA and/or RNA from a variety of samples are well-known in the art.

As used herein, “labels” are chemical or biochemical moieties useful for labeling a nucleic acid (including a single nucleotide), amino acid, or antibody. “Labels” include fluorescent agents, chemiluminescent agents, chromogenic agents, quenching agents, radionuclides, enzymes, substrates, cofactors, inhibitors, magnetic particles, and other moieties known in the art. “Labels” are capable of generating a measurable signal and may be covalently or noncovalently joined to an oligonucleotide or nucleotide.

The term “multiplex PCR” as used herein refers to an assay that provides for simultaneous amplification of two or more products within the same reaction vessel. Each product is primed using a distinct primer pair. A multiplex reaction may further include labeled primers each product, that are detectably labeled with different detectable moieties.

As used herein, the term “oligonucleotide” refers to a short polymer composed of deoxyribonucleotides, ribonucleotides or any combination thereof. Oligonucleotides are generally between about 10, 11, 12, 13, 14, or 15 to about 150 nucleotides (nt) in length, more preferably about 10, 11, 12, 13, 14, or 15 to about 25, 30, 35, 40, 50, or 70 nt, and most preferably between about 18 to about 26 nt in length. The single letter code for nucleotides is as described in the U.S. Patent Office Manual of Patent Examining Procedure, section 2422, table 1. In this regard, the nucleotide designation “R” means purine such as guanine or adenine, “Y” means pyrimidine such as cytosine or thymidine (uracil if RNA); and “M” means adenine or cytosine. An oligonucleotide may be used as a primer or as a probe.

As used herein, a “primer” for amplification is an oligonucleotide that is complementary to a target nucleotide sequence and leads to addition of nucleotides to the 3′ end of the primer in the presence of a DNA or RNA polymerase. The 3′ nucleotide of the primer should generally be identical to the target sequence at a corresponding nucleotide position for optimal expression and/or amplification. The term “primer” as used herein includes all forms of primers that may be synthesized including peptide nucleic acid primers, locked nucleic acid primers, phosphorothioate modified primers, labeled primers, and the like.

An oligonucleotide (e.g., a probe or a primer) that is specific for a target nucleic acid will “hybridize” to the target nucleic acid under suitable conditions. As used herein, “hybridization” or “hybridizing” refers to the process by which an oligonucleotide single strand anneals with a complementary strand through base pairing under defined hybridization conditions. Oligonucleotides used as primers or probes for specifically amplifying (i.e., amplifying a particular target nucleic acid sequence) or specifically detecting (i.e., detecting a particular target nucleic acid sequence) a target nucleic acid generally are capable of specifically hybridizing to the target nucleic acid.

“Specific hybridization” is an indication that two nucleic acid sequences share a high degree of complementarity. Specific hybridization complexes form under permissive annealing conditions and remain hybridized after any subsequent washing steps. Permissive conditions for annealing of nucleic acid sequences are routinely determinable by one of ordinary skill in the art and may occur, for example, at 65° C. in the presence of about 6×SSC Stringency of hybridization may be expressed, in part, with reference to the temperature under which the wash steps are carried out. Such temperatures are typically selected to be about 5° C. to 20° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Equations for calculating Tₘ and conditions for nucleic acid hybridization are known in the art.

As used herein, a primer is “specific” for a nucleic acid if the oligonucleotide has at least 50% sequence identity with a portion of the nucleic acid when the oligonucleotide and the nucleic acid are aligned. A primer that is specific for a nucleic acid is one that, under the appropriate hybridization or washing conditions, is capable of hybridizing to the target of interest and not substantially hybridizing to nucleic acids which are not of interest. Higher levels of sequence identity are preferred and include at least 75%, at least 80%, at least 85%, at least 90%, at least 95% and more preferably at least 98% sequence identity. Sequence identity can be determined using a commercially available computer program with a default setting that employs algorithms well known in the art (e.g., BLAST). As used herein, sequences that have “high sequence identity” have identical nucleotides at least at about 50% of aligned nucleotide positions, preferably at least at about 60% of aligned nucleotide positions, and more preferably at least at about 75% of aligned nucleotide positions.

As used herein, the term “sample” or “biological sample” may comprise clinical samples, isolated nucleic acids, or isolated microorganisms. In preferred embodiments, a sample is obtained from a biological source (i.e., a “biological sample”), such as tissue, bodily fluid, or microorganisms collected from a subject. Sample sources include, but are not limited to, sputum (processed or unprocessed), bronchial alveolar lavage (BAL), bronchial wash (BW), nasopharyngeal swabs (NP), nasopharyngeal aspirates, blood, stool, bodily fluids, cerebrospinal fluid (CSF), urine, plasma, serum, or tissue (e.g., biopsy material).

The terms “target nucleic acid” or “target sequence” as used herein refer to a sequence which includes a segment of nucleotides of interest to be amplified and detected. Copies of the target sequence which are generated during the amplification reaction are referred to as amplification products, amplimers, or amplicons. Target nucleic acid may be composed of segments of a chromosome, a complete gene with or without intergenic sequence, segments or portions of a gene with or without intergenic sequence, or sequence of nucleic acids which probes or primers are designed. Target nucleic acids may include a wild-type sequence(s), a mutation, deletion or duplication, tandem repeat regions, a gene of interest, a region of a gene of interest or any upstream or downstream region thereof. Target nucleic acids may represent alternative sequences or alleles of a particular gene. Target nucleic acids may be derived from genomic DNA, cDNA, or RNA. As used herein target nucleic acid may be DNA or RNA extracted from a cell or a nucleic acid copied or amplified therefrom.

### Detection of Target Nucleic Acids

In one aspect, the methods can be used to detect the presence of a single target nucleic acid. For example, a single primer containing a labeled 5′ tail region may be used in conjunction with a universal quenching oligonucleotide (an “anti-primer”) that is complementary to the tail region. Under suitable conditions, the label of the primer is capable of generating a detectable signal. In one embodiment, the anti-primer comprises an interactive label that quenches the signal from the label on the free, unincorporated primer.

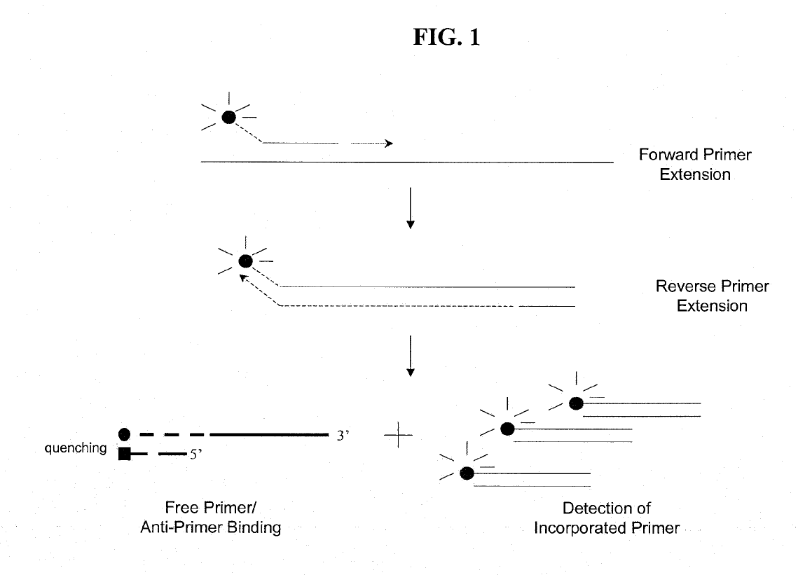

In one embodiment, the primer comprising a labeled 5′ tail region and a target-specific region is contacted with a sample to be tested for the presence of a target nucleic acid under conditions where the primer hybridizes to the target and is extended by a DNA polymerase enzyme (See FIG. 1). An unlabeled primer may also be included in the reaction in order to amplify the genetic target of interest using PCR (FIG. 1). This primer may be a “reverse” primer where the labeled primer is the “forward” primer. Alternatively, the unlabeled primer may be a “forward” primer where the “reverse” primer is the labeled primer. Following primer extension from the unlabeled primer, a 5′ end-labeled fluorescent double-stranded amplification product is produced.

Next, the temperature of the reaction mixture is lowered to less than the Tₘ of the duplex formed between the anti-primer and the 5′ tail region of the primer, which may be less than about 60° C., less than about 55° C., less than about 50° C., less than about 45° C., or less than about 40° C. The Tₘ for the primer/anti-primer duplex will depend on the sequence of nucleotides within the region. Equations for calculating Tₘ and conditions for nucleic acid hybridization are known in the art. Lowering the temperature allows the anti-primer to hybridize to and quench the signal from the free (unincorporated) labeled primer (FIG. 1). The fluorescence of the amplification product is then recorded. Thus, the polymerase synthesis step is de-coupled from the signal detection step. When a primer is incorporated into an amplification product, its signal cannot be quenched by the anti-primer because it is incorporated into a double-stranded structure. Therefore, the amount of signal from the label is correlated to the amount of amplification product. The methods provide real time monitoring of the absolute gene dosage of a genetic target of interest.

In one embodiment, the methods can be used to detect two or more target nucleic acids in a single reaction, i.e., a multiplex amplification. A primer containing a labeled 5′ tail region is provided for each genetic target of interest. In suitable embodiments, the label of each primer is different and the signals from each label may be read simultaneously. The 5′ tail region of each primer is complementary to a labeled anti-primer, so that under suitable hybridization conditions, the signal from the label on each of the primers may be quenched by the label on the anti-primer. When a primer is incorporated in an amplification product, its signal cannot be quenched by the anti-primer because it is incorporated into a double-stranded structure. Therefore, the amount of signal from each label is correlated to the amount of each amplification product. Accordingly, the methods provide for simultaneous real time monitoring of the absolute gene dosage of a multiple genetic targets.

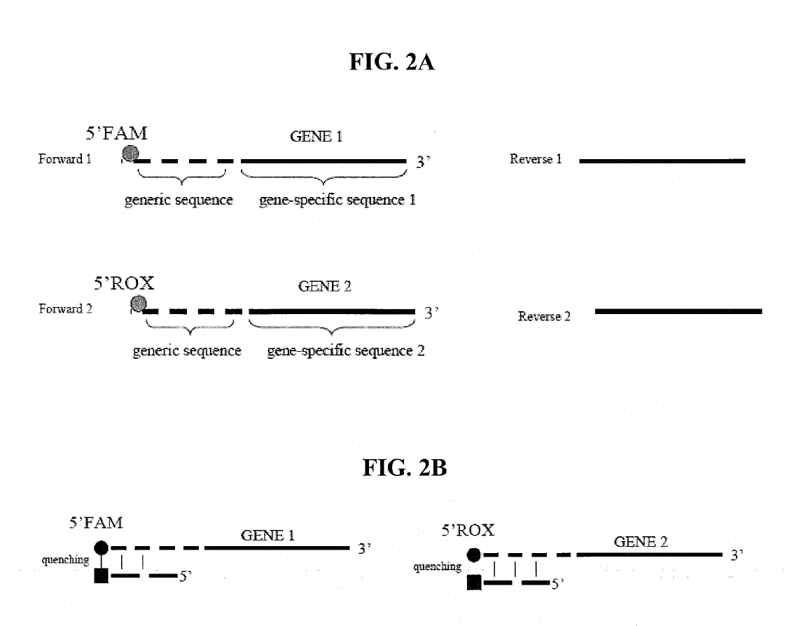

In an illustrative embodiment, shown in FIG. 2, the primers and anti-primers for multiplex detection of two target nucleic acids are shown. The principle of multiplex amplification is similar to the single target embodiment described above. Thus, one or more sets of primers are designed to have a universal, 5′-positioned oligonucleotide tail fluorescently labeled with a different fluorophore for each target gene (FIG. 2A). An anti-primer carrying a label (e.g., a 3′-BHQ) that quenches all unincorporated fluorescent primers simultaneously is also included in the PCR reaction (FIG. 2B). Next, the temperature of the reaction mixture is lowered below the Tₘ of the primer/anti-primer duplex, e.g., by approximately 5° C. to 10° C. This allows the anti-primer to hybridize to and quench the signal from the free labeled primer because the anti-primer has a lower melting temperature (Tₘ) than the primer and is complementary to the primer tail. The fluorescence of the PCR product is then recorded. Both genetic targets 1 and 2 can be quantified simultaneously. In some embodiments, multiple target nucleic acids, e.g., 2 or more, 3 or more, 4 or more, 5 or more, or 6 or more target nucleic acids, can be detected in a single reaction, with each primer incorporating a different detectable label. The number of targets that may be detected depends on the capability of the instrument to read multiple signals simultaneously.

In another aspect, the methods are capable of detecting the dosage difference between any two genomic regions, or between two alleles of the same gene that are modified via mutation, deletion or methylation. In one embodiment, the methods are used to detect a minority of altered target nucleic acids in a large population of wild-type nucleic acids. Often it is not possible to identify a small number of cells that harbor gene dosage changes within a large excess of wild type cells. Thus, in particular embodiments, the methods described herein are useful for the sensitive, early detection of gene dosage, allelic imbalance or methylation imbalance in cancer cells.

In one embodiment, two target nucleic acids (e.g. targets 1 and 2) can be made to generate fluorescent PCR products that combine and counter-act each other's fluorescent signal during amplification. Where approximately equal numbers of genetic region 1 and 2 are present, amplification produces very low (or zero) signal. However, if there is a dosage difference between genetic target regions 1 and 2 (e.g. target 1 has more copies than target 2), amplification generates a strong signal that is proportional to the amount of dosage difference. In this manner the dosage difference between two genomic regions present only in a minority of the cell population is amplified and can be resolved even in the presence of a very large excess of cells that have no dosage difference between targets regions 1 and 2 (i.e. wild type cells).

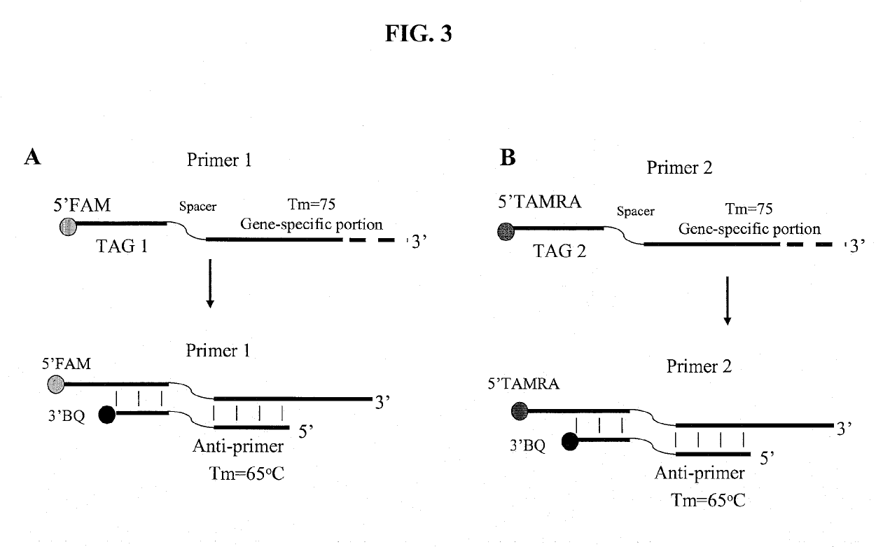

In one embodiment, the primers specific for the target nucleic acids are designed to contain a polymerase blocking group or spacer, which cannot be replicated by the polymerase enzyme (FIG. 3). Attached to the 5′ end of the polymerase blocking group on each primer are labeled tail sequences (shown in FIG. 3 as TAG1 and TAG2). Both tail sequences may be labeled with fluorophores that quench each other when in close proximity (FIG. 3). The tail regions of the primers (TAG1 and TAG2) are complementary to each other and designed to hybridize at a temperature lower than the temperature of the amplification reaction. Anti-primers containing quenchers that bind to the free unincorporated primers are also included in the reaction (FIG. 3).

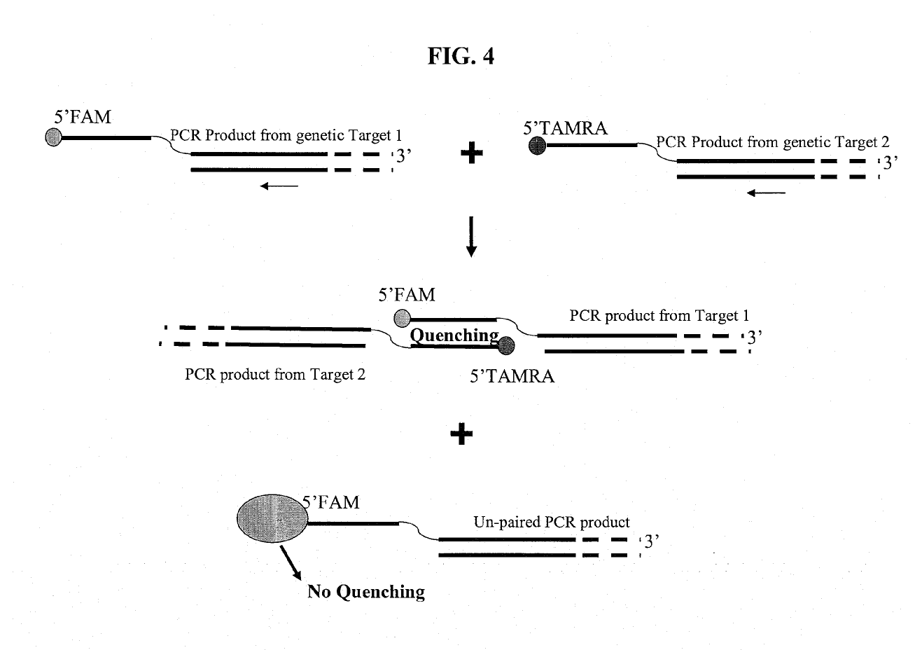

During PCR, fluorescent PCR products corresponding to the dosage of the target nucleic acids are produced simultaneously (FIG. 4). Following primer extension, the temperature is lowered to less than the Tₘ, of the 5′ tail regions, which may be less than about 60° C., less than about 55° C., less than about 50° C., less than about 45° C., or less than about 40° C. The hybridization temperature for the 5′ tail regions will depend on the sequence of nucleotides within the region. Equations for calculating Tₘ and conditions for nucleic acid hybridization are known in the art. When the temperature is lowered, the anti-primers bind to free primers and quench their fluorescence; and the fluorescent PCR products bind to each other on a one-to-one basis due to the complementarity of the tail portions of the primers. Upon hybridization, the fluorescence of each amplification product is strongly-quenched due to the proximity of the fluorophores. Only tail regions of amplification products from either target 1 or target 2 that are in excess dosage remain unhybridized at the lowered temperature. These unhybridized products fluoresce and their fluorescence is indicative of the dosage difference between the two genetic targets of interest.

In one embodiment, fluorescent PCR products corresponding to the dosage of genetic target 1 and 2 are produced (FIG. 4). By lowering the temperature below the Tₘ of the anti-primers, the fluorescence of the free primers is quenched by hybridization to their respective anti-primers. By lowering the temperature even more, below the Tₘ of the primer tails TAG1 and TAG2 the fluorescent PCR products bind to each other due to the complementarity of TAG1 and TAG2 portions of the primers and the FAM and TAMRA fluorescence is self-quenched due to the proximity of the fluorophores. Only PCR products from either genetic target 1 or genetic target 2 that are in excess dosage of each other remain unhybridized at the lowered temperature. These unhybridized products fluoresce strongly and their fluorescence is indicative of the dosage difference between the two genetic targets of interest. The Tₘ chosen for primers, anti-primers and TAGS is flexible. In one embodiment, the Tₘ of the duplex between the primers and the target will be highest; and the Tₘ of the duplex between the free primer and the antiprimer will be higher than the hybridization temperature of the two primer tails. In some embodiments, the range of Tₘ for primers can be from about 60° C. to 80° C.; the range of Tₘ for the antiprimers can be from about 50° C. to 70° C.; and the range of Tₘ for the tail sequences (TAG1 and TAG2) can be from about 30° C. to 60° C.

Accordingly, when equal amounts of target nucleic acids for genetic region 1 and 2 are present, PCR produces very low (or zero) signal. However, if there is a dosage difference between genetic target regions 1 and 2 (e.g., genetic target 1 has more copies than genetic target 2), PCR generates a strong signal that is proportional to the amount of dosage difference. In this manner the dosage difference between two genomic regions present only in a minority of the cell population is amplified and can be resolved even in the presence of a very large excess of cells that have no dosage difference between genetic regions 1 and 2 (i.e. wild type cells).

In one embodiment, the amount of the two target nucleic acids can be determined by performing a melt procedure in which the hybridized product of the tails (TAG1 and TAG2) of primer 1 and primer 2 (FIG. 3) are separated by increasing the temperature of the reaction while monitoring the fluorescent signal. As the products separate, the fluorescent signals from the labels on TAG1 and TAG2 will increase in proportion to the amount of product present that was hybridized. This signal, in addition to the dosage difference signal, can be used to determine the amount of target nucleic acids present in the sample.

### Design of Labeled Primers and Anti-Primers

In various embodiments, the methods of the invention utilize labeled primers and anti-primers for the detection of target nucleic acids. In one embodiment, a primer is provided which is suitable for amplifying a target nucleic acid. The primer typically has a 3′ target-specific region and a 5′ tail region. The 3′ target-specific region is complementary to the target nucleic acid and is suitable for amplifying the target in a nucleic acid amplification reaction. The 5′ tail consists of a sequence added to the 5′-end of the forward or, alternatively, the reverse gene-specific primer, depending on which placement is less likely to contain secondary (hairpin) structures, as predicted by Oligo 6 or similar software. The length of the tail sequence may be from about 5 to 30, about 10 to 25, about 10 to 20, or about 15 to 20 nucleotides. In one embodiment, the Tₘ of the tail region is less than the Tₘ of the target-specific region, e.g., at least about 5° C. less, at least about 7° C. less, at least about 10° C. less, or at least about 15° C. less. In one embodiment, the tail sequence is about 17 nucleotides and has a Tₘ of approximately 57° C. and the target-specific region has a Tm of approximately 65° C., as calculated by Oligo 6 or similar software. In a particular embodiment, the tail has a sequence according to SEQ ID NO: 2 and the anti-primer has a sequence according to SEQ ID NO: 1.

In certain embodiments, the primer may further comprise a linker or spacer moiety, which prevents polymerase mediated chain extension on the primer template. This polymerase blocking group may be placed between the target-specific region and the 5′ tail region of the primer. Such spacers are well known and include, but are not limited to, a deoxyribose chain that lacks the bases (i.e. a chain of abasic sites known as C-3, C-5, C-9, or C-15 depending on whether there is a string of 3, 5, 9 or 15 sugar molecules involved, respectively), and a string of modified nucleotides that allows hybridization but does not allow DNA polymerase synthesis, for example iso-guanine nucleotide or iso-cytosine nucleotides. In one embodiment, the polymerase blocking group may include hexethylene glycol (HEG) monomer. Alternatively, the linker may comprise material such as 2-O-alkyl RNA which will not permit polymerase mediated replication of a complementary strand.

In one embodiment, the linker further comprises nucleotides not complementary to the target nucleic acid. Optimum characteristics for the linker may be determined by routine experimentation. The linker may comprise less than 200 nucleotides, less than 100 nucleotides, less than 50 nucleotides, or less than 20 nucleotides. In suitable embodiment, the linker comprises HEG as the polymerase blocking group and less than 20 nucleotides.

In some embodiments, an anti-primer is provided which is complementary to the 5′ tail region of the primer. The anti-primer complementary to this tail also has Tₘ less than the Tₘ of the target-specific portion of the primer. In a particular embodiment, the annealing-extension portion of the PCR reaction is conducted at approximately 60° C. At this temperature, the primers can anneal to the target and may be extended without interference from the anti-primer, which has a lower Tₘ Following primer extension, the temperature is lowered to approximately 50° C. and the anti-primer anneals to the tail region of the free, single-stranded primer, but not the double-stranded PCR product.

In one embodiment, labeled primers are provided in order to determine the dosage difference between two target nucleic acids. The tail regions of the primers are complementary to each other and designed such that at a lower temperature the PCR products from targets 1 and 2 hybridize to each other and quench strongly each other's fluorescent signal. In one embodiment, the labels on two primer are the same, so that they self-quench when in close proximity, e.g., within about 0-50 Angstroms of each other. Anti-primers are also included in the reaction (FIG. 3). The anti-primers may also contain a spacer similar to the one designed for the primers and are labeled with a quencher moiety. In one embodiment, the anti-primers are substantially complementary to part of the 5′ tail and part of the gene-specific portions of the respective primers.

In one embodiment, the concentration of the anti-primer is from about 2 to 3 times, about 2 to 4 times, about 2 to 5 times, or about 2 to 10 times that of the primer. Accordingly, the majority of the free primer is expected to bind the anti-primer under suitable conditions (i.e., lowered temperature), thus strongly quenching the primer fluorescence. Since the 5′-end of the primer-tail is in opposite orientation to the 3′-end of the anti-primer, the interaction is mediated via excitation interaction (Bernacchi and Mely 2001; Bernacchi, Piemont et al. 2003) between the 5′-fluorophore and the 3′-quencher present on the tail and anti-primer, respectively, which for most fluorophores provides stronger quenching than fluorescence resonance energy transfer (FRET) (Marras, Kramer et al. 2002). Careful design of primers using appropriate software minimizes the probability for secondary structures and primer-dimer formation in aQRT-PCR. Further, in some embodiments, the anti-primer will not participate in primer-dimer formation since the placement of the quenching molecule on the 3′ end of the anti-primer will be an effective polymerase block (Holland, Abramson et al. 1991).

The primers and anti-primers described herein may comprise a label. Nucleotides and oligonucleotides can be labeled by incorporating moieties detectable by spectroscopic, photochemical, biochemical, immunochemical, or chemical assays. The method of linking or conjugating the label to the nucleotide or oligonucleotide depends on the type of label(s) used and the position of the label on the nucleotide or oligonucleotide.

A variety of labels which are appropriate for use in the methods are disclosed herein and are known in the art. These include, but are not limited to, fluorescent dyes, chromophores, chemiluminescent labels, electrochemiluminescent labels, such as ORI-TAG™ (Igen), ligands having specific binding partners, or any other labels that can interact with each other to enhance, alter, or diminish a signal. It is understood that, should the PCR be practiced using a thermocycler instrument, a label should be selected to survive the temperature cycling required in this automated process.

In some embodiments, the primers and anti-primers used in the methods are labeled. For example, the oligonucleotides may include a label that emits a detectable signal. By way of example, the label system may be used to produce a detectable signal based on a change in fluorescence, fluorescence resonance energy transfer (FRET), fluorescence quenching, phosphorescence, bioluminescence resonance energy transfer (BRET), or chemiluminescence resonance energy transfer (CRET).

In some embodiments, two interactive labels may be used on a single oligonucleotide with due consideration given for maintaining an appropriate spacing of the labels on the oligonucleotide to permit the separation of the labels during oligonucleotide hydrolysis. In other embodiments, two interactive labels on different oligonucleotides may be used, such as, for example, the anti-primer and the tail region of the primer. In this embodiment, the anti-primer and the tail region are designed to hybridize to each other. Consideration is given to having an appropriate spacing of the labels between the oligonucleotides when hybridized.

The oligonucleotides and nucleotides of the disclosed methods may be labeled with a “fluorescent dye” or a “fluorophore.” As used herein, a “fluorescent dye” or a “fluorophore” is a chemical group that can be excited by light to emit fluorescence. Some suitable fluorophores may be excited by light to emit phosphorescence. Dyes may include acceptor dyes that are capable of quenching a fluorescent signal from a fluorescent donor dye. Dyes that may be used in the disclosed methods include, but are not limited to, the following dyes and/or dyes sold under the following tradenames: 1,5 IAEDANS; 1,8-ANS; 4-Methylumbelliferone; 5-carboxy-2,7-dichlorofluorescein; 5-Carboxyfluorescein (5-FAM); 5-Carboxytetramethylrhodamine (5-TAMRA); 5-HAT (Hydroxy Tryptamine); 5-Hydroxy Tryptamine (HAT); 5-ROX (carboxy-X-rhodamine); 6-Carboxyrhodamine 6G; 66-JOE; 7-Amino-4-methylcoumarin; 7-Aminoactinomycin D (7-AAD); 7-Hydroxy-4-methylcoumarin; 9-Amino-6-chloro-2-methoxyacridine; ABQ; Acid Fuchsin; ACMA (9-Amino-6-chloro-2-methoxyacridine); Acridine Orange; Acridine Red; Acridine Yellow; Acriflavin; Acriflavin Feulgen SITSA; Alexa Fluor 350™; Alexa Fluor 430™; Alexa Fluor 488™; Alexa Fluor 532™; Alexa Fluor 546™; Alexa Fluor 568™; Alexa Fluor 594™; Alexa Fluor 633™; Alexa Fluor 647™; Alexa Fluor 660™; Alexa Fluor 680™; Alizarin Complexon; Alizarin Red; Allophycocyanin (APC); AMC; AMCA-S; AMCA (Aminomethylcoumarin); AMCA-X; Aminoactinomycin D; Aminocoumarin; Aminomethylcoumarin (AMCA); Anilin Blue; Anthrocyl stearate; APC (Allophycocyanin); APC-Cy7; APTS; Astrazon Brilliant Red 4G; Astrazon Orange R; Astrazon Red 6B; Astrazon Yellow 7 GLL; Atabrine; ATTO-TAG™ CBQCA; ATTO-TAG™ FQ; Auramine; Aurophosphine G; Aurophosphine; BAO 9 (Bisaminophenyloxadiazole); Berberine Sulphate; Beta Lactamase; BFP blue shifted GFP (Y66H); Blue Fluorescent Protein; BFP/GFP FRET; Bimane; Bisbenzamide; Bisbenzimide (Hoechst); Blancophor FFG; Blancophor SV; BOBO™-1; BOBO™-3; Bodipy 492/515; Bodipy 493/503; Bodipy 500/510; Bodipy 505/515; Bodipy 530/550; Bodipy 542/563; Bodipy 558/568; Bodipy 564/570; Bodipy 576/589; Bodipy 581/591; Bodipy 630/650-X; Bodipy 650/665-X; Bodipy 665/676; Bodipy FL; Bodipy FL ATP; Bodipy Fl-Ceramide; Bodipy R6G SE; Bodipy TMR; Bodipy TMR-X conjugate; Bodipy TMR-X, SE; Bodipy TR; Bodipy TR ATP; Bodipy TR-X SE; BO-PRO™-1; BO-PRO™-3; Brilliant Sulphoflavin FF; Calcein; Calcein Blue; Calcium Crimson™; Calcium Green; Calcium Orange; Calcofluor White; Carboxy-X-rhodamine (5-ROX); Cascade Blue™; Cascade Yellow; Catecholamine; CCF2 (GeneBlazer); CFDA; CFP—Cyan Fluorescent Protein; CFP/YFP FRET; Chlorophyll; Chromomycin A; CL-NERF (Ratio Dye, pH); CMFDA; Coelenterazine f; Coelenterazine fcp; Coelenterazine h; Coelenterazine hcp; Coelenterazine ip; Coelenterazine n; Coelenterazine O; Coumarin Phalloidin; C-phycocyanine; CPM Methylcoumarin; CTC; CTC Formazan; Cy2™; Cy3.1 8; Cy3.5™; Cy3™; Cy5.1 8; Cy5.5™; Cy5™; Cy7™; Cyan GFP; cyclic AMP Fluorosensor (FiCRhR); Dabcyl; Dansyl; Dansyl Amine; Dansyl Cadaverine; Dansyl Chloride; Dansyl DHPE; Dansyl fluoride; DAPI; Dapoxyl; Dapoxyl 2; Dapoxyl 3; DCFDA; DCFH (Dichlorodihydrofluorescein Diacetate); DDAO; DHR (Dihydrorhodamine 123); Di-4-ANEPPS; Di-8-ANEPPS (non-ratio); DiA (4-Di-16-ASP); Dichlorodihydrofluorescein Diacetate (DCFH); DiD—Lipophilic Tracer; DiD (DiIC18(5)); DIDS; Dihydrorhodamine 123 (DHR); DiI (DiIC18(3)); Dinitrophenol; DiO (DiOC18(3)); DiR; DiR (DiIC18(7)); DNP; Dopamine; DsRed; DTAF; DY-630-NHS; DY-635-NHS; EBFP; ECFP; EGFP; ELF 97; Eosin; Erythrosin; Erythrosin ITC; Ethidium Bromide; Ethidium homodimer-1 (EthD-1); Euchrysin; EukoLight; Europium (III) chloride; EYFP; Fast Blue; FDA; Feulgen (Pararosaniline); FITC; Flazo Orange; Fluo-3; Fluo-4; Fluorescein (FITC); Fluorescein Diacetate; Fluoro-Emerald; Fluoro-Gold (Hydroxystilbamidine); Fluor-Ruby; Fluor X; FM 1-43™; FM 4-46; Fura Red™; Fura Red™/Fluo-3; Fura-2; Fura-2/BCECF; Genacryl Brilliant Red B; Genacryl Brilliant Yellow 10GF; Genacryl Pink 3G; Genacryl Yellow 5GF; GeneBlazer (CCF2); GFP (S65T); GFP red shifted (rsGFP); GFP wild type, non-UV excitation (wtGFP); GFP wild type, UV excitation (wtGFP); GFPuv; Gloxalic Acid; Granular Blue; Haematoporphyrin; Hoechst 33258; Hoechst 33342; Hoechst 34580; HPTS; Hydroxycoumarin; Hydroxystilbamidine (FluoroGold); Hydroxytryptamine; Indo-1; Indodicarbocyanine (DiD); Indotricarbocyanine (DiR); Intrawhite Cf; JC-1; JO-JO-1; JO-PRO-1; Laurodan; LDS 751 (DNA); LDS 751 (RNA); Leucophor PAF; Leucophor SF; Leucophor WS; Lissamine Rhodamine; Lissamine Rhodamine B; Calcein/Ethidium homodimer; LOLO-1; LO-PRO-1; Lucifer Yellow; Lyso Tracker Blue; Lyso Tracker Blue-White; Lyso Tracker Green; Lyso Tracker Red; Lyso Tracker Yellow; LysoSensor Blue; LysoSensor Green; LysoSensor Yellow/Blue; Mag Green; Magdala Red (Phloxin B); Mag-Fura Red; Mag-Fura-2; Mag-Fura-5; Mag-Indo-1; Magnesium Green; Magnesium Orange; Malachite Green; Marina Blue; Maxilon Brilliant Flavin 10 GFF; Maxilon Brilliant Flavin 8 GFF; Merocyanin; Methoxycoumarin; Mitotracker Green FM; Mitotracker Orange; Mitotracker Red; Mitramycin; Monobromobimane; Monobromobimane (mBBr-GSH); Monochlorobimane; MPS (Methyl Green Pyronine Stilbene); NBD; NBD Amine; Nile Red; NED™; Nitrobenzoxadidole; Noradrenaline; Nuclear Fast Red; Nuclear Yellow; Nylosan Brilliant Iavin EBG; Oregon Green; Oregon Green 488-X; Oregon Green™; Oregon Green™ 488; Oregon Green™ 500; Oregon Green™ 514; Pacific Blue; Pararosaniline (Feulgen); PBFI; PE-Cy5; PE-Cy7; PerCP; PerCP-Cy5.5; PE-TexasRed [Red 613]; Phloxin B (Magdala Red); Phorwite AR; Phorwite BKL; Phorwite Rev; Phorwite RPA; Phosphine 3R; Phycoerythrin B [PE]; Phycoerythrin R [PE]; PKH26 (Sigma); PKH67; PMIA; Pontochrome Blue Black; POPO-1; POPO-3; PO-PRO-1; PO-PRO-3; Primuline; Procion Yellow; Propidium Iodid (PI); PyMPO; Pyrene; Pyronine; Pyronine B; Pyrozal Brilliant Flavin 7GF; QSY 7; Quinacrine Mustard; Red 613 [PE-TexasRed]; Resorufin; RH 414; Rhod-2; Rhodamine; Rhodamine 110; Rhodamine 123; Rhodamine 5 GLD; Rhodamine 6G; Rhodamine B; Rhodamine B 200; Rhodamine B extra; Rhodamine BB; Rhodamine BG; Rhodamine Green; Rhodamine Phallicidine; Rhodamine. Phalloidine; Rhodamine Red; Rhodamine WT; Rose Bengal; R-phycocyanine; R-phycoerythrin (PE); RsGFP; S65A; S65C; S65L; S65T; Sapphire GFP; SBFI; Serotonin; Sevron Brilliant Red 2B; Sevron Brilliant Red 4G; Sevron Brilliant Red B; Sevron Orange; Sevron Yellow L; sgBFP™; sgBFP™ (super glow BFP); sgGFP™; sgGFP™ (super glow GFP); SITS; SITS (Primuline); SITS (Stilbene Isothiosulphonic Acid); SNAFL calcein; SNAFL-1; SNAFL-2; SNARF calcein; SNARF1; Sodium Green; SpectrumAqua; SpectrumGreen; SpectrumOrange; Spectrum Red; SPQ (6-methoxy-N-(3-sulfopropyl)quinolinium); Stilbene; Sulphorhodamine B can C; Sulphorhodamine G Extra; SYTO 11; SYTO 12; SYTO 13; SYTO 14; SYTO 15; SYTO 16; SYTO 17; SYTO 18; SYTO 20; SYTO 21; SYTO 22; SYTO 23; SYTO 24; SYTO 25; SYTO 40; SYTO 41; SYTO 42; SYTO 43; SYTO 44; SYTO 45; SYTO 59; SYTO 60; SYTO 61; SYTO 62; SYTO 63; SYTO 64; SYTO 80; SYTO 81; SYTO 82; SYTO 83; SYTO 84; SYTO 85; SYTOX Blue; SYTOX Green; SYTOX Orange; TET™; Tetracycline; Tetramethylrhodamine (TRITC); Texas Red™; Texas Red-X™ conjugate; Thiadicarbocyanine (DiSC3); Thiazin Red R; Thiazole Orange; Thioflavin 5; Thioflavin S; Thioflavin TCN; Thiolyte; Thiozole Orange; Tinopol CBS (Calcofluor White); TMR; TO-PRO-1; TO-PRO-3; TO-PRO-5; TOTO-1; TOTO-3; TriColor (PE-Cy5); TRITC TetramethylRodamineIsoThioCyanate; True Blue; TruRed; Ultralite; Uranine B; Uvitex SFC; VIC®; wt GFP; WW 781; X-Rhodamine; XRITC; Xylene Orange; Y66F; Y66H; Y66W; Yellow GFP; YFP; YO-PRO-1; YO-PRO-3; YOYO-1; YOYO-3; and salts thereof.

Fluorescent dyes or fluorophores may include derivatives that have been modified to facilitate conjugation to another reactive molecule. As such, fluorescent dyes or fluorophores may include amine-reactive derivatives such as isothiocyanate derivatives and/or succinimidyl ester derivatives of the fluorophore.

The primers of the present methods may be labeled with a donor fluorophore and an acceptor fluorophore (or quencher dye) that are present in the oligonucleotides at positions that are suitable to permit FRET (or quenching). In some embodiments, the primers and/or anti-primers of the disclosed methods may be labeled with a quencher. The quenching molecule can be placed at any position along the primer or anti-primer, which allows the flexibility to achieve ideal quenching that results in strong signal generation. Interactive labels may utilize proximal quenching or FRET quenching. In proximal quenching (a.k.a. “contact” or “collisional” quenching), the donor is in close proximity to the quencher moiety such that energy of the donor is transferred to the quencher, which dissipates the energy as heat as opposed to a fluorescence emission. In FRET quenching, the donor fluorophore transfers its energy to a quencher which releases the energy as fluorescence at a higher wavelength. Proximal quenching requires very close positioning of the donor and quencher moiety, while FRET quenching, also distance related, occurs over a greater distance (generally 1-10 nm, the energy transfer depending on R⁻⁶, where R is the distance between the donor and the acceptor). Thus, when FRET quenching is involved, the quenching moiety is an acceptor fluorophore that has an excitation frequency spectrum that overlaps with the donor emission frequency spectrum. When quenching by FRET is employed, the assay may detect an increase in donor fluorophore fluorescence resulting from increased distance between the donor and the quencher (acceptor fluorophore) or a decrease in acceptor fluorophore emission resulting from decreased distance between the donor and the quencher (acceptor fluorophore). Examples of donor/acceptor dye pairs for FRET are known in the art and may include fluorophores and quenchers described herein such as Fluorescein/Tetramethylrhodamine, IAEDANS™/Fluorescein (Molecular Probes, Eugene, Oreg.), EDANS™/Dabcyl, Fluorescein/Fluorescein (Molecular Probes, Eugene, Oreg.), BODIPY™ FL/BODIPY™ FL (Molecular Probes, Eugene, Oreg.), and Fluorescein/QSY7™.

In some embodiments, the quencher molecule is a molecule that absorbs transferred energy but does not emit fluorescence, e.g., “a dark quencher.” In many embodiments, the dark quencher has maximum absorbance of between about 400 and about 700 nm, and often between about 500 and about 600 nm. In certain embodiments, the dark quencher comprises a substituted 4-(phenyldiazenyl)phenylamine structure, often comprising at least two residues selected from aryl, substituted aryl, heteroaryl, substituted heteroaryl and combination thereof, wherein at least two of said residues are covalently linked via an exocyclic diazo bond. Suitable quenchers include Dabcyl or dark quenchers such as, Iowa Black™, or black hole quenchers sold under the tradename “BHQ” (e.g., BHQ-0, BHQ-1, BHQ-2, and BHQ-3, Biosearch Technologies, Novato, Calif.). Dark quenchers also may include quenchers sold under the tradename “QXL™” (Anaspec, San Jose, Calif.) or DNP-type non-fluorophores that include a 2,4-dinitrophenyl group.

The labels can be attached to the oligonucleotides directly or indirectly by a variety of techniques. Depending upon the precise type of label used, the label can be located at the 5′ or 3′ end of the primer or anti-primer, located internally in the primer or anti-primer's nucleotide sequence, or attached to spacer arms extending from the primer or anti-primer and having various sizes and compositions to facilitate signal interactions. Using commercially available phosphoramidite reagents, one can produce oligonucleotides containing functional groups (e.g., thiols or primary amines) at either terminus, for example by the coupling of a phosphoramidite dye to the 5′ hydroxyl of the 5′ base by the formation of a phosphate bond, or internally, via an appropriately protected phosphoramidite, and can label them using protocols described in, for example, PCR Protocols: A Guide to Methods and Applications, ed. by Innis et al., Academic Press, Inc., 1990. Methods for incorporating oligonucleotide functionalizing reagents having one or more sulfhydryl, amino or hydroxyl moieties into the oligonucleotide reporter sequence, typically at the 5′ terminus, are described in U.S. Pat. No. 4,914,210, incorporated herein by reference. Labels at the 3′ terminus, for example, can employ polynucleotide terminal transferase to add the desired moiety, such as for example, cordycepin, ³⁵S-dATP, and biotinylated dUTP.

The label of the primer or antiprimer can be positioned at any suitable location. In one embodiment, the labeled primer(s) and anti-primer comprise a pair of interactive signal-generating labels effectively positioned on the primer and on the anti-primer (or on a second primer) so as to quench the generation of detectable signal when the interactive signal-generating labels are in sufficiently close proximity to each other. Examples of such labels include dye/quencher pairs or two dye pairs (where the emission of one dye stimulates emission by the second dye).

In one embodiment, the interactive signal generating pair comprises a fluorophore and a quencher that can quench the fluorescent emission of the fluorophore. For example, a quencher may include a BHQ and the fluorophore may be FAM or ROX. Other fluorophore-quencher pairs have been described in Morrison, Detection of Energy Transfer and Fluorescence Quenching in Nonisotopic Probing, Blotting and Sequencing, Academic Press, 1995.

### Nucleic Acid Amplification

In one embodiment, the nucleic acid amplification is performed in a real-time homogeneous assay. A real-time assay is one that produces data indicative of the presence or quantity of a target molecule during the amplification process, as opposed to the end of the amplification process. A homogeneous assay is one in which the amplification and detection reagents are mixed together and simultaneously contacted with a sample, which may contain a target nucleic acid molecule. Thus, the ability to detect and quantify DNA targets in real-time homogeneous systems as amplification proceeds is centered in single-tube assays in which the processes required for target molecule amplification and detection take place in a single “closed-tube” reaction format.

Homogenous PCR methods (closed tube methods) offer the advantage that they do not require the operator to perform manual separation of the amplified target by means of gel electrophoresis or other methods. Once setup is complete, target detection can be accomplished without additional manipulation of the sample. Such assays facilitate high throughput by monitoring the accumulation of fluorescence in a closed tube. Once the sample extract and reagents are combined, the tube is sealed and does not need to be opened again. This method minimizes the likelihood of false-positive results due to carryover contamination of the sample (a notable shortcoming of many nucleic acid amplification-based detection systems), facilitates sample tracking, and significantly reduces hands-on processing time.

In various embodiments, a polymerase enzyme is used in the amplification of nucleic acids. Suitable nucleic acid polymerases include, for example, polymerases capable of extending an oligonucleotide by incorporating nucleic acids complementary to a template oligonucleotide. For example, the polymerase can be a DNA polymerase. Enzymes having polymerase activity catalyze the formation of a bond between the 3′ hydroxyl group at the growing end of a nucleic acid primer and the 5′ phosphate group of a nucleotide triphosphate. These nucleotide triphosphates are usually selected from deoxyadenosine triphosphate (A), deoxythymidine triphosphate (T), deoxycytosine triphosphate (C) and deoxyguanosine triphosphate (G).

Because the relatively high temperatures necessary for strand denaturation during methods such as PCR can result in the irreversible inactivation of many nucleic acid polymerases, nucleic acid polymerase enzymes useful for performing the methods disclosed herein preferably retain sufficient polymerase activity to complete the reaction when subjected to the temperature extremes of methods such as PCR. Typically, the nucleic acid polymerase enzymes useful for the methods disclosed herein are thermostable nucleic acid polymerases. Suitable thermostable nucleic acid polymerases include, but are not limited to, enzymes derived from thermophilic organisms. Examples of thermophilic organisms from which suitable thermostable nucleic acid polymerase can be derived include, but are not limited to, Thermus aquaticus, Thermus thermophilus, Thermus flavus, Thermotoga neapolitana and species of the Bacillus, Thermococcus, Sulfobus, and Pyrococcus genera. Nucleic acid polymerases can be purified directly from these thermophilic organisms. However, substantial increases in the yield of nucleic acid polymerase can be obtained by first cloning the gene encoding the enzyme in a multicopy expression vector by recombinant DNA technology methods, inserting the vector into a host cell strain capable of expressing the enzyme, culturing the vector-containing host cells, then extracting the nucleic acid polymerase from a host cell strain which has expressed the enzyme. Suitable thermostable nucleic acid polymerases, such as those described above, are commercially available.

In addition, it will be recognized that RNA can be used as a sample and that a reverse transcriptase can be used to transcribe the RNA to cDNA. The transcription can occur prior to or during PCR amplification. Examples of reverse transcriptases that can be used include, but are not limited to, ImProm-II Reverse Transcriptase (Promega, Madison, Wis.) and BD Powerscript Reverse Transcriptase (BD Biosciences, Franklin Lakes, N.J.). Methods for using reverse transcriptases to prepare and obtain cDNA molecules are well known in the art and are described in Sambrook, J. et al., Molecular Cloning: A Laboratory Manual, 2nd edition, Cold Spring Harbor Press, Cold Spring Harbor, N.Y. (1989).

In a suitable embodiment, real time PCR is performed using any suitable instrument capable of detecting fluorescence from one or more fluorescent labels. For example, real time detection on the instrument (e.g. a ABI Prism® 7900HT sequence detector) monitors fluorescence and calculates the measure of reporter signal, or Rn value, during each PCR cycle. The threshold cycle, or Ct value, is the cycle at which fluorescence intersects the threshold value. The threshold value is determined by the sequence detection system software or manually.

In some embodiments, melting curve analysis may be used to detect an amplification product. Melting curve analysis involves determining the melting temperature of an nucleic acid amplicon by exposing the amplicon to a temperature gradient and observing a detectable signal from a fluorophore. Melting curve analysis is based on the fact that a nucleic acid sequence melts at a characteristic temperature called the melting temperature (Tₘ), which is defined as the temperature at which half of the DNA duplexes have separated into single strands. The melting temperature of a DNA depends primarily upon its nucleotide composition. Thus, DNA molecules rich in G and C nucleotides have a higher Tₘ than those having an abundance of A and T nucleotides.

Where a fluorescent dye is used to determine the melting temperature of a nucleic acid in the method, the fluorescent dye may emit a signal that can be distinguished from a signal emitted by any other of the different fluorescent dyes that are used to label the oligonucleotides. In some embodiments, the fluorescent dye for determining the melting temperature of a nucleic acid may be excited by different wavelength energy than any other of the different fluorescent dyes that are used to label the oligonucleotides. In some embodiments, the second fluorescent dye for determining the melting temperature of the detected nucleic acid is an intercalating agent. Suitable intercalating agents may include, but are not limited to SYBR™ Green 1 dye, SYBR™ dyes, Pico Green, SYTO dyes, SYTOX dyes, ethidium bromide, ethidium homodimer-1, ethidium homodimer-2, ethidium derivatives, acridine, acridine orange, acridine derivatives, ethidium-acridine heterodimer, ethidium monoazide, propidium iodide, cyanine monomers, 7-aminoactinomycin D, YOYO-1, TOTO-1, YOYO-3, TOTO-3, POPO-1, BOBO-1, POPO-3, BOBO-3, LOLO-1, JOJO-1, cyanine dimers, YO-PRO-1, TO-PRO-1, YO-PRO-3, TO-PRO-3, TO-PRO-5, PO-PRO-1, BO-PRO-1, PO-PRO-3, BO-PRO-3, LO-PRO-1, JO-PRO-1, and mixture thereof. In suitable embodiments, the selected intercalating agent is SYBR™ Green 1 dye.

By detecting the temperature at which the fluorescence signal is lost, the melting temperature can be determined. In the disclosed methods, each of the amplified target nucleic acids may have different melting temperatures. For example, each of these amplified target nucleic acids may have a melting temperature that differs by at least about 1° C., more preferably by at least about 2° C., or even more preferably by at least about 4° C. from the melting temperature of any of the other amplified target nucleic acids. By observing differences in the melting temperature(s) of the respective amplification products, one can confirm the presence or absence of the target nucleic acids in the sample.

To minimize the potential for cross contamination, reagent and mastermix preparation, specimen processing and PCR setup, and amplification and detection are all carried out in physically separated areas.

### EXAMPLES

The present invention is further illustrated by the following examples, which should not be construed as limiting in any way.

### Example 1

### Materials and Methods for Anti-Primer Quenching Real-Time PCR

Reference human male genome DNA was purchased from Promega (Madison, Wis.). BT474 genomic DNA was purified from cultured BT474 breast cancer cell line obtained from the American Tissue Culture Collection (Manassas, Va.). The 51 human surgical lung tissue samples were obtained from the Massachusetts General Hospital Tumor Bank. The four breast cancer stage Her2-positive (Her2+) samples were obtained from the Dana Farber Cancer Center SPORE Bank following manual microdissection. The formalin-fixed paraffin-embedded (FFPE) specimens were obtained from the Department of Pathology, Brigham and Women's Hospital. Plasma samples were obtained from the Medical Oncology Tumor Bank, Dana Farber Cancer Institute. The use of unidentifiable human specimens for genetic analysis was approved by the Institutional Review Board.

Genomic DNA from cell line and fresh tissues was extracted and purified with the DNAeasy Kit™ (Qiagen, Valencia, Calif.). A modified method was used for extraction of DNA from FFPE. Briefly, 25 mg of tissue per sample was deparaffinized by treatment with mixed xylenes (1.2 ml, vortexed, centrifuged 3 min at RT, remove xylene and repeat 1-2× until clear), and xylenes removed by addition of 100% ethanol (1.2 ml, vortex, centrifuge 3 min at RT, remove ETON and repeat 1-2× until clear). Following vaporization of ethanol for 10 min at 37° C., samples were washed in PBS (1.2 ml, vortex, centrifuge 3 min at RT, remove PBS). Tissue was placed in 360 μl of lysis buffer (Qiagen)+40 μl PK and rotated at 55° for 24-72 hours as needed for full digestion. Subsequent DNA purification was carried out using the DNAeasy kit, adjusting buffer and extraction volumes for the volume of lysis buffer used. Quality of extracted DNA was initially evaluated by gel electrophoresis of 0.75 μg DNA in a 1% agarose gel. To extract plasma-circulating DNA, within 2-5 hours of collection, whole blood was centrifuged at 2000×g for 15-30 min and plasma was carefully collected from the top of the supernatant, as described in Li, Harris et al. 2006. Plasma-circulating DNA was purified from plasma with QlAamp™ MinElute Virus spin kit (Qiagen, Valencia, Calif.) and quantified using the PicoGreen™ method (Molecular Probes, Eugene, Oreg.).

Nucleic acid amplification was performed using the AmpliTaq Gold™ amplification kit (Applied Biosystems, Branchburg, N.J.) in a Smart-Cycler™ real time thermocycler (Cepheid, Sunnyvale, Calif.). The labeled and non-labeled primers were designed with Oligo 6 software (Molecular Biology Insights, Cascade, Colo.) and synthesized by Integrated DNA technologies (Coralville, Iowa). The sequences of the primers and anti-primer are shown in Table 1. 6-FAM (FAM) or ROX-NHS-Ester (ROX) were used as labels at the 5′ end of the forward or, alternatively, the reverse fluorescent primer. Serial dilutions of DNA (0.14 to 145 ng) in a 1 μl volume were added to a final volume of 20 μl with a final concentration of 1×ABI TaqMan master mix (Applied Biosystems), 0.2 μM each fluorescence labeled primer, unlabeled primer and μM BHQ-2-labeled anti-primer (Table 1) (synthesized by Integrated DNA technologies). The thermocycling program was 50° C. 2 min 1 cycle, 95° C. for 10 min 1 cycle, and 40-50 cycles (95° C. for 15 sec; 60° C. for 30 sec; 50° C. for 30 sec and 50° C. 15 sec for reading fluorescence).

TABLE 1
Primers and probes for aQRT-PCR

| | | |
|---|---|---|
| Gene | Sequence (5′→3′) | SEQ ID NO: |

| | | | |
|---|---|---|---|
| Antiprimer | | TTCCCTCGGATAGCACT | SEQ ID NO: 1 |
| Primer Tail | | AGTGCTATCCGAGGGAA | SEQ ID NO: 2 |
| (TAG1) | | | |
| HER-2 | Forward | GGATGTGCGGCTCGTACAC | SEQ ID NO: 3 |
| | Reverse | FAM-AGTGCTATCCGAGGGAATGACATGG | SEQ ID NO: 4 |
| | | TTGGGACTCTTGAC | |
| GAPDH | Forward | ROX-GTGCTATCCGAGGGAACCTGACCTG | SEQ ID NO: 5 |
| | | CCGTCTAGAAAA | |
| | Reverse | CTCCGACGCCTGCTTCAC | SEQ ID NO: 6 |
| TOP1 | Forward | FAM-AGTGCTATCCGAGGGAAGACAGCCC | SEQ ID NO: 7 |
| (103 bp) | | CGGATGAGAAC | |
| | Reverse | AAGAATTGCAACAGCTCGATTG | SEQ ID NO: 8 |
| HBEGF | Forward | FAM-AGTGCTATCCGAGGGAACCCCAGTT | SEQ ID NO: 9 |
| (99 bp) | | GCCGTCTAGGA | |
| | Reverse | CGGACATACTCTGTTTGGCACTT | SEQ ID NO: 10 |
| TBP | Forward | FAM-AGTGCTATCCGAGGGAAGGGCATTA | SEQ ID NO: 11 |
| (108 bp) | | TTTGTGCACTGAGA | |
| | Reverse | AGCAGCACGGTATGAGCAACTGTCAGA | SEQ ID NO: 12 |
| MYC | Forward | FAM-AGTGCTATCCGAGGGAATCCTCCTT | SEQ ID NO: 13 |
| (134 bp) | | ATGCCTCTATCAT | |
| | Reverse | CCGCGCTTTGATCAAGAGTCC | SEQ ID NO: 14 |

### Example 2

### Simplex Anti-Primer-Based Quantitative Real-Time PCR

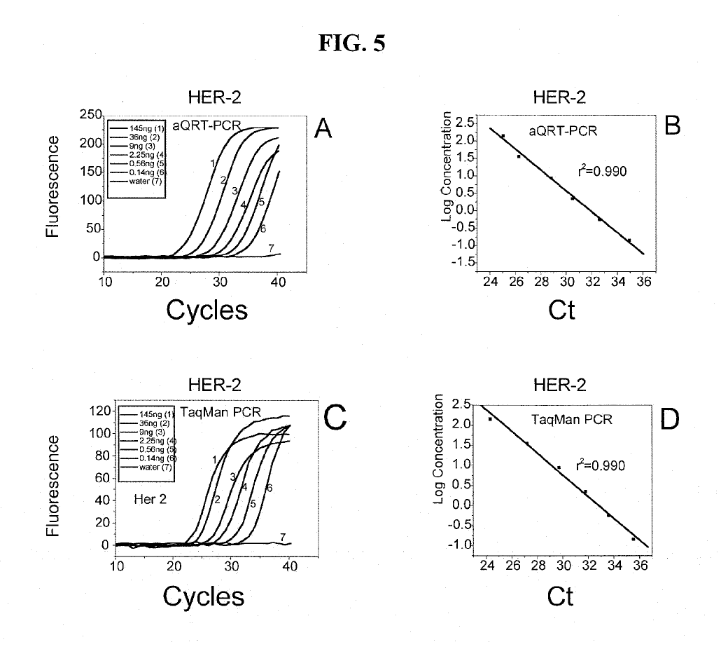

An aQRT-PCR approach was used for simplex amplification and quantification of several genes from human genomic DNA. Amplification conditions were as described in Example 1. For the target nucleic acid HER-2, primary growth curves were obtained using varying amounts of starting genomic reference DNA down to an equivalent of 20 cells (FIG. 5A). A standard curve (log concentration versus threshold cycle) is shown in FIG. 5B.

For comparison, the TaqMan® real time PCR method was performed in parallel (FIGS. 5C and 5D). The two methods utilized the same PCR kit, annealing and extension temperature, fluorophore and quencher. Under essentially identical PCR parameters, aQRT-PCR generated significantly stronger fluorescence signals than TaqMan® (FIG. 5), which reflects the stronger quenching effected by direct contact of FAM and BHQ-2 in aQRT-PCR, compared to TaqMan's fluorescence energy transfer (FRET). The data also demonstrate that the two methods have a similar Pearson correlation coefficient (r²), indicating their equivalency for simplex HER-2 quantification.

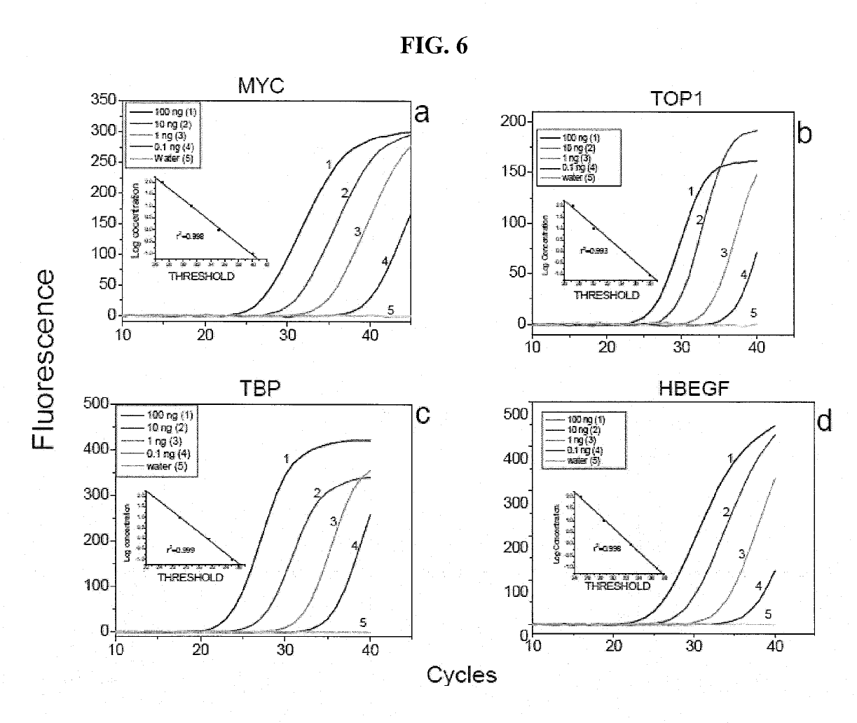

Four additional genes (TBP, MYC, HBEGF and TOP1) with varying amplicon sizes (69 by to 134 bp) were tested using simplex aQRT-PCR performed in triplicate independent experiments. Representative primary growth curves are depicted in FIG. 6. The results demonstrate strong signals and linear log-concentration-versus-Ct curves (r²>0.99) while the input genomic DNA limit is about 20 cell equivalents (approximately 0.1 ng genomic DNA). The no-DNA controls (water) do not demonstrate signals for at least 40-45 PCR cycles for the primers tested.

### Example 3

### Multiplex aQRT-PCR of HER-2 and GAPDH Target Nucleic Acids

Multiplex aQRT-PCR was performed using FAM-labeled reverse and ROX-labeled forward primers for the HER-2 oncogene and the GAPDH housekeeping gene, respectively, in order to quantify HER-2 amplification in a single tube reaction. Serial dilutions of DNA (0.14-145 ng) in a 1 μl volume from Reference or BT474 cells was added to a final volume of 20 μl containing a final concentration of 1×ABI TaqMan® master mix (Applied Biosystems), 0.05 μM each FAM-labeled HER-2 reverse primer and unlabeled HER-2 forward primer, 0.15 μM each ROX-labeled GAPDH forward primer and unlabeled GAPDH reverse primer, 1 μM BHQ-labeled anti-primer. The thermocycling program was 50° C. 2 min 1 cycle, 95° C. for 10 min 1 cycle, and 40 cycles (95° C. for 15 sec; 60° C. for 30 sec; 50° C. for 30 sec and 50° C. 15 sec for reading fluorescence). Fluorescence was read in both FAM and ROX channels simultaneously. Three independent experiments were performed for each gene to generate an average relative copy number and standard deviation. The relative gene amplification between unamplified/amplified plasma-circulating DNA was calculated using the comparative threshold (ΔΔCt) method (Heid 1996; Wang, Brennan et al. 2004).

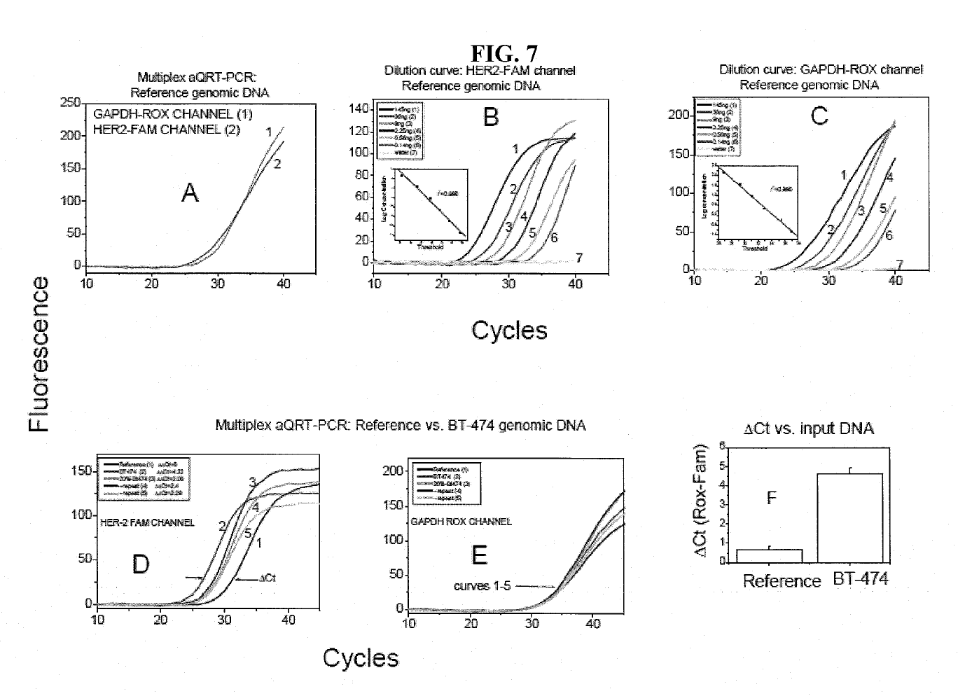

For an optimal co-amplification of HER-2 and GAPDH, the ratio of FAM and ROX-labeled primers was experimentally determined to be 1:3. Under these conditions the two genes are amplified with similar amplification efficiency when reference genomic DNA is used (FIG. 7A). Next, serial dilution of the starting material, human male reference genomic DNA (0.14 to 145 ng) was tested, and the linearity of the multiplex aQRT-PCR response was observed on both channels simultaneously (FIGS. 7B and 7C). The multiplex assay was linear (r²=0.995) down to a starting material equivalent to 20 cells, while the negative control (water) was negative in both FAM and ROX channels for at least 45 PCR cycles.

The ability of the multiplex assay to quantify, in a single reaction, the established amplification of the oncogene HER-2 in genomic DNA from BT-474 breast cancer cells is demonstrated in FIGS. 7D and 7E. The GAPDH-normalized threshold difference (ΔΔCt=4.2) is in good agreement to that obtained when the simplex TaqMan assay, in two separate reactions (ΔΔCt=3.9), for BT-474 genomic DNA, as described in Wang, Maher et al. 2004. Furthermore, in triplicate repeated experiments, a 20% dilution of BT-474 DNA within reference DNA was reliably discriminated from pure reference DNA (curves 3-5 in FIGS. 7D and 7E). This indicates that the method should have the precision to detect a 20% minority of HER-2 amplified cancer cells within 80% stromal cells.

Finally, ΔΔCt for BT-474 cells was examined when the starting genomic DNA material is gradually reduced from 145 ng down to 0.14 ng. The results shown in FIG. 7F indicate that ΔΔCt remains substantially constant even at low input DNA (relative standard deviation of ΔΔCt is ±13%), indicating the ability of the multiplex approach to reliably quantify gene amplification in minute DNA samples obtained from fine needle biopsy or from tissue microdissection.

HER-2 Amplification in Clinical Samples Detected by Multiplex aQRT-PCR

HER-2 is over-expressed 20-30% of breast cancers (Harris, Liotcheva et al. 2001), ovarian cancer (Slamon, Godolphin et al. 1989) and other cancers (Scholl, Beuzeboc et al. 2001; Nathanson, Culliford et al. 2003), and is correlated with clinical outcome (Harris, Liotcheva et al. 2001). To demonstrate the utility of multiplex aQRT-PCR detection of HER-2 amplification in fresh DNA from microdissected clinical samples, HER-2 amplification was tested from DNA extracted from 4 manually-dissected breast cancer specimens characterized as HER-2 positive by immunohistochemistry (IHC) and FISH approaches (Harris, Liotcheva et al. 2001). To examine clinical samples for HER-2 amplification using multiplex aQRT-PCR, 2 ng genomic DNA from the microdissected breast cancer samples (Her2+) was used in the reaction. For the FFPE samples, 20 ng was used as input genomic DNA. For the plasma-circulating DNA samples, 1 μl from each Qiagen-purified DNA sample was added to the reaction.

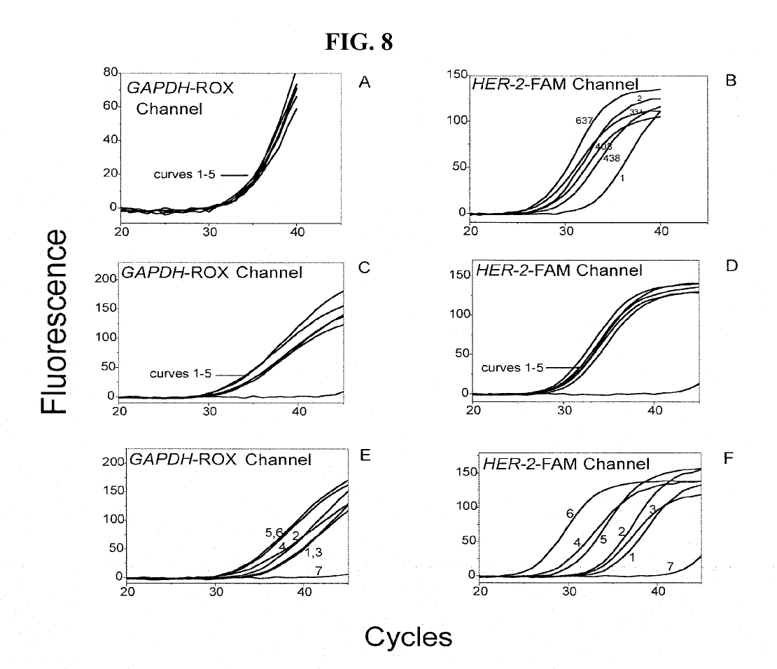

FIGS. 8A and 8B demonstrate primary growth curves for the four samples using a starting DNA amount of 2 ng (equivalent to approximately 350 cells) along with reference and BT-474 DNA using the multiplex aQRT-PCR for HER-2/GAPDH. All four samples were shown to harbor substantial (>8-fold) chromosomal HER-2 amplification, in agreement with the FISH and IHC determinations. The threshold difference (ΔΔCt) for the four samples ranged between approximately 3 and 5 cycles, similar to the amplification detected in BT-474 breast cancer cells (Microdissected sample #334, ΔΔCt=5.5; Microdissected sample #438, ΔΔCt=2.9; Microdissected sample #637, ΔΔCt=5.8; Microdissected sample #408, ΔΔCt=3.5 Reference DNA, ΔΔCt=O; BT474 DNA, ΔΔCt=4.1).

To examine the utility of the method in situations where the starting DNA material is of low quality and/or quantity, we applied multiplex aQRT-PCR to the detection of HER-2 in DNA from formalin-fixed-paraffin-embedded (FFPE) specimens, as well as in free-circulating DNA extracted from the plasma of colon and ovarian cancer patients. DNA extracted from these clinical samples is highly fragmented and is often difficult to amplify (Lehmann and Kreipe 2001; Wang, Maher et al. 2004; Li, Harris et al. 2005; Li, Harris et al. 2006). FIGS. 8C and 8D demonstrate multiplex HER-2/GAPDH amplification using DNA from FFPE samples (20 ng each) obtained from glioma cancer patients that harbor significant DNA degradation due to the formalin fixation procedure. Compared to the threshold obtained for the reference DNA (10 ng DNA) in the same experiment, aQRT-PCR was not significantly affected by the fragmentation in the starting material cycles (FFPE #2, ΔΔCt=0.7; FFPE #3, ΔΔCt=1.4; FFPE #19, ΔΔCt=1.0; FFPE #56, ΔΔCt=0.1).

Next, multiplex HER-2/GAPDH amplification from four plasma-circulating DNA samples donated from colon and ovarian cancer patients was conducted. In this case, 1 μl purified DNA was used in each reaction, and experiments were repeated two independent times. One of the plasma samples obtained from a colon cancer patient (sample #4) harbors a ˜6-fold HER-2 amplification, while the remaining plasma samples are negative for amplification. The results are depicted in FIGS. 8E and 8F and indicate that, for short amplicons like those used for HER-2 and GAPDH (approximately 70 by each), multiplex aQRT-PCR is not significantly affected by the fragmentation status of the input material.

### Example 4

### Real-Time Simplex and Multiplex SNP-Genotyping Via aQRT-PCR

To adapt aQRT-PCR for real-time SNP genotyping, allele-specific PCR based on a 3′-mismatched nucleotide (Newton, Graham et al. 1989; Sommer, Cassady et al. 1989) was used. A well-studied polymorphism of the apolipoprotein B gene (B71, C>T) was selected for validation of the method. The published allele-specific PCR primers of the B71 single nucleotide polymorphism (C>T) of human apolipoprotein-B (Germer and Higuchi 1999) were adapted by adding different fluorescent probes to the tails of each allele. The ROX-labeled forward primer, specific for the C genotype, was: 5′-ROX-AGTGCTATCCGAGGGAAGAAGACCAGCCAGTGCAC (SEQ ID NO: 15); the FAM-labeled forward primer, specific for the T genotype, was: 5′-FAM-AGTGCTATCCGAGGGAATGAAGACCAGCCAGTGCAT (SEQ ID NO:16); and the reverse primer was 5′-CAAGGCTTTGCCCTCAGGGTT (SEQ ID NO:17).

The PCR reaction was conducted in 20 μl volume using a Smart Cycler™ real time PCR machine. The real time PCR reaction was set-up as follows: 40 ng human genome DNA from clinical lung samples, 0.2 μM each C- or T-genotype-specific forward primer; 0.2 p.M reverse primer; 1× Stoffel polymerase buffer; an extra 30 mM KCl to final concentration of 40 mM; 2 mM MgCl₂; 50 μM each dATP, dCTP, dGTP, and dTTP; 5% DMSO; 2.5% glycerol and 2 units of Stoffel Taq polymerase (Perkin Elmer, Wellesley, Mass.). The themocycling program was 95° C. for 2 min 1 cycle, and 40 cycles (95° C. for 15 sec; 60° C. for 30 sec; 50° C. for 30 sec and 50° C. 15 sec for simultaneous reading of fluorescence from FAM and ROX channels). For multiplex genotyping, the AmpliTaq Gold™ (Applied Biosystems) was used instead of the Stoffel Taq polymerase. Multiplex real time PCR to genotype B71 SNP was performed in 20 μl final volume with a final concentration of 1×ABI TaqMan master mix (Applied Biosystems), 40 ng genomic DNA, 0.05 μM FAM-labeled T-specific primer, 0.15 μM ROX-labeled C-specific primer, 0.2 μM unlabeled reverse primer, and 1 μM BHQ2-labeled anti-primer. The themocycling program was: 50° C. 2 min 1 cycle, 95° C. for 10 min 1 cycle, and 40 cycles (95° C. for 15 sec; 60° C. for 30 sec; 50° C. for 30 sec and 50° C. 15 sec for reading fluorescence from FAM and ROX channels). To determine the reproducibility of the multiplex aQRT-PCR approach for genotype determination, the experiments were repeated 10 independent times.

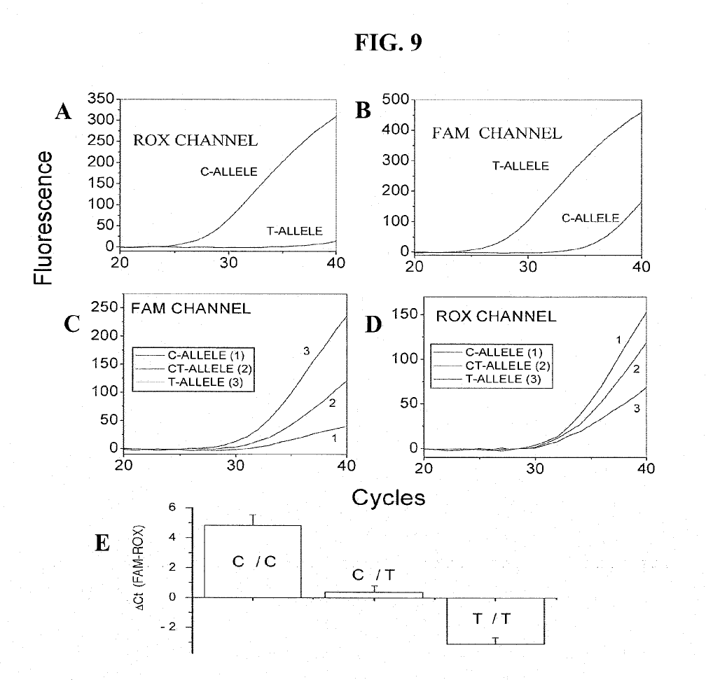

Two DNA samples previously sequenced at the Dana Farber Core sequencing facility and known to be homozygous C/C and T/T were first tested via simplex aQRT-PCR using the Stoffel fragment of Taq polymerase. FIGS. 9A and 9B demonstrate an 8-cycle threshold difference between the two alleles, corresponding to an approximate 256-fold discrimination. Next, the method was applied in a multiplex format using Stoffel Taq polymerase, but the reaction yield was suboptimal (data not shown). Therefore, a multiplex aQRT-PCR using the Amplitaq Gold polymerase was employed. For multiplex aQRT-PCR, the ratio of FAM- and ROX-labeled allele-specific primers yielding optimal signals for both alleles was experimentally determined to be 1:3. FIGS. 9C and 9D demonstrate multiplex SNP-genotyping in three genomic DNA samples (40 ng each) containing the C/C (homozygous), C/T (heterozygous) and TT (homozygous) genotypes. The genotype of these three samples was verified via sequencing. To examine the reproducibility of the multiplex approach, the experiment was repeated another 9 independent times, and the average ΔCt and standard deviation (SD) are depicted in FIG. 9E. There is a 4.5±0.7 cycle (C/C) and a 3.5±0.4 cycle (T/T) threshold difference between each of these two homozygous samples and the heterozygous (C/T) sample. Assuming Poison statistics, the threshold range covered by [ΔCt±3SD] is expected to cover 99.73% of the distribution of values (Obuchowski 1998). Accordingly, multiplex aQRT-PCR is able to determine the three apolipoprotein B genotypes with high confidence.

### Multiplex SNP Genotyping of Clinical Samples

To validate further the use of multiplex aQRT-PCR in clinical samples, the method was used to determine the apolipoprotein B genotype in genomic DNA extracted from 51 surgical lung specimens. Duplicate independent experiments were carried out using multiplex aQRT-PCR and the average ΔCt (ROX-FAM) was calculated. Genotype was determined by comparing ΔCt to the [ΔCt±3SD] range of genotype-specific thresholds derived from the experiment in FIG. 9C. In parallel, the DNA was submitted for sequencing. The results of this study indicated a complete (51/51) agreement between the two independent methods (Table 2).

TABLE 2
Comparison of genotyping results obtained
by aQRT-PCR and sequencing.

| Genotype | Samples, n | Concordance |
|---|---|---|
| C | 27 | 27/27 |
| CT | 18 | 18/18 |
| T | 6 | 6/6 |
| Total | 51 | 51/51 |

Thus, it should be understood that although the present invention has been specifically disclosed by preferred embodiments and optional features, modification, improvement and variation of the inventions embodied therein herein disclosed may be resorted to by those skilled in the art, and that such modifications, improvements and variations are considered to be within the scope of this invention. The materials, methods, and examples provided here are representative of preferred embodiments, are exemplary, and are not intended as limitations on the scope of the invention.

In addition, where features or aspects of the invention are described in terms of Markush groups, those skilled in the art will recognize that the invention is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

Other embodiments are set forth within the following claims.

### REFERENCES

Bernacchi, S, and Y. Mely (2001). “Exciton interaction in molecular beacons: a sensitive sensor for short range modifications of the nucleic acid structure.” Nucleic Acids Res 29(13): E62-2.
Bernacchi, S., E. Piemont, et al. (2003). “Excitonic heterodimer formation in an HIV-1 oligonucleotide labeled with a donor-acceptor pair used for fluorescence resonance energy transfer.” Biophys J 84(1): 643-54.
Eads, C. A., K. D, Danenberg, et al. (2000). “MethyLight: a high-throughput assay to measure DNA methylation.” Nucleic Acids Res 28(8): E32.
Germer, S, and R. Higuchi (1999). “Single-tube genotyping without oligonucleotide probes.” Genuine Res 9(1): 72-8.
Harris, L. N., V. Liotcheva, et al. (2001). “Comparison of methods of measuring HER-2 in metastatic breast cancer patients treated with high-dose chemotherapy.” J Clin Oncol 19(6): 1698-706.
Heid, C., Stevens, J., Livak, K., Williams, P. (1996). Real time quantitative PCR. Genome Methods Genome Research. Cold Spring Harbor, N.Y., Cold Spring Harbor Laboratory Press.
Herman, J. G., J. R. Graff, et al. (1996). “Methylation-specific PCR: a novel PCR assay for methylation status of CpG islands.” Proc Natl Acad Sci USA 93(18): 9821-6.
Holland, P. M., R. D. Abramson, et al. (1991). “Detection of specific polymerase chain reaction product by utilizing the 5′ to 3′ exonuclease activity of Thermus aquaticus DNA polymerase.” Proc Natl Acad Sci USA 88(16): 7276-80.
Jirtle, R. L., M. Sander, et al. (2000). “Genomic imprinting and environmental disease susceptibility.” Environ Health Perspect 108(3): 271-8.
Kallioniemi, A., O. P. Kallioniemi, et al. (1994). “Detection and mapping of amplified DNA sequences in breast cancer by comparative genomic hybridization.” Proc Natl Acad Sci USA 91(6): 2156-60.
Kinzler, K. W. and B. Vogelstein (1996). “Lessons from hereditary colorectal cancer.” Cell 87(2): 159-70.
Lehmann, U. and H. Kreipe (2001). “Real-time PCR analysis of DNA and RNA extracted from formalin-fixed and paraffin-embedded biopsies.” Methods 25(4): 409-18.
Li, J., L. Harris, et al. (2005). “Whole genome amplification of plasma-circulating DNA enables expanded screening for allelic imbalance in plasma.” Journal of Molecular Diagnostics, In Press.
Li, J., L. Harris, et al. (2006). “Whole genome amplification of plasma-circulating DNA enables expanded screening for allelic imbalance in plasma.” Journal of Molecular Diagnostics 8(1): 22-30.
Li, J., F. Wang, et al. (2006). “Antiprimer quenching-based real-time PCR and its application to the analysis of clinical cancer samples.” Clinical Chemistry 52(4): 624-33.
Lin, N. U., G. Broadwater, et al. (2004). “The predictive value of HER2 and p53 on outcomes after paclitaxel chemotherapy for metastatic breast cancer: Results from CALGB 9342.” Journal of Clinical Oncology 22(14S): 9562.
Marras, S. A., F. R. Kramer, et al. (2002). “Efficiencies of fluorescence resonance energy transfer and contact-mediated quenching in oligonucleotide probes.” Nucleic Acids Res 30(21): e122.
Nathanson, D. R., A. T. t. Culliford, et al. (2003). “HER 2/neu expression and gene amplification in colon cancer.” Int J Cancer 105(6): 796-802.
Nazarenko, I., B. Lowe, et al. (2002). “Multiplex quantitative PCR using self-quenched primers labeled with a single fluorophore.” Nucleic Acids Res 30(9): e37.
Nazarenko, I. A., S. K. Bhatnagar, et al. (1997). “A closed tube format for amplification and detection of DNA based on energy transfer.” Nucleic Acids Res 25(12): 2516-21.
Newton, C. R., A. Graham, et al. (1989). “Analysis of any point mutation in DNA. The amplification refractory mutation system (ARMS).” Nucleic Acids Res 17(7): 2503-16.
Obuchowski, N. A. (1998). “Sample size calculations in studies of test accuracy.” Stat Methods Med Res 7(4): 371-92.
Paez, J. G., P. A. Janne, et al. (2004). “EGFR mutations in lung cancer: correlation with clinical response to gefitinib therapy.” Science 304(5676): 1497-500.
Scholl, S., P. Beuzeboc, et al. (2001). “Targeting HER2 in other tumor types.” Ann Oncol Suppl 1: S81-7.
Slamon, D. J., W. Godolphin, et al. (1989). “Studies of the HER-2/neu proto-oncogene in human breast and ovarian cancer.” Science 244(4905): 707-12.
Sommer, S. S., J. D. Cassady, et al. (1989). “A novel method for detecting point mutations or polymorphisms and its application to population screening for carriers of phenylketonuria.” Mayo Clin Proc 64(11): 1361-72.
Sommer, S. S., A. R. Groszbach, et al. (1992). “PCR amplification of specific alleles (PASA) is a general method for rapidly detecting known single-base changes.” Biotechniques 12(1): 82-7.
Todd, A. V., C. J. Fuery, et al. (2000). “DzyNA-PCR: use of DNAzymes to detect and quantify nucleic acid sequences in a real-time fluorescent format.” Clin Chem 46(5): 625-30.
Tyagi, S, and F. R. Kramer (1996). “Molecular beacons: probes that fluoresce upon hybridization.” Nat Biotechnol 14(3): 303-8.
Wang, G., C. Brennan, et al. (2004). “Balanced-PCR amplification allows unbiased identification of genomic copy changes in minute cell and tissue samples.” Nucleic Acids Res 32(9): e76.
Wang, G., E. Maher, et al. (2004). “DNA amplification method tolerant to sample degradation.” Genome Res 14(11): 2357-66.
Whitcombe, D., J. Theaker, et al. (1999). “Detection of PCR products using self-probing amplicons and fluorescence.” Nat Biotechnol 17(8): 804-7.
Wittwer, C. T., M. G. Hellmann, et al. (1997). “Continuous fluorescence monitoring of rapid cycle DNA amplification.” Biotechniques 22(1): 130-1, 134-8.
Zhang, Y., D. Zhang, et al. (2003). “A novel real-time quantitative PCR method using attached universal template probe.” Nucleic Acids Res 31(20): e123.

What is claimed is:

1. A method comprising:
(a) contacting a sample to be tested for the presence of a target nucleic acid with (i) a primer comprising a first label and a first and a second region of nucleotides, wherein the first region of nucleotides is complementary to the target nucleic acid; and (ii) an anti-primer comprising a second label and a nucleotide sequence complementary to the second region of the primer, wherein the second label is capable of quenching a detectable signal from the first label, and
further wherein the sample is contacted under conditions wherein the primer specifically hybridizes to the target nucleic acid, if present in the sample;
(b) performing an amplification reaction with the primer to produce an amplification product having an incorporated primer; and
(c) detecting the presence of the target nucleic acid in the sample by detecting the first label of the incorporated primer under conditions wherein the anti-primer specifically hybridizes to the second region of the primer and hybridization between the primer and the anti-primer quenches the detectable signal from unincorporated primer.

2. The method of claim 1, wherein the first and second label comprise a fluorophore/quencher pair.

3. The method of claim 1, wherein the first label is a fluorophore and the second label is a quencher.

4. The method of claim 3, wherein the fluorophore is selected from the group consisting of FAM, TAMRA, ROX, Cy5, Cy3, and BODIPY, and the quencher is a dark quencher.

5. The method of claim 1, wherein the melting temperature of the first region of the first primer is higher than the melting temperature of the anti-primer.

6. The method of claim 5, wherein the melting temperature of the first region of the first primer is from 5 to 10 degrees Celsius higher than the melting temperature of the anti-primer.

7. The method of claim 1, wherein the detecting the first label comprises lowering the temperature of the reaction below the melting temperature of the anti-primer and measuring the signal from the first label.

8. The method of claim 1, wherein the sample of step (a) is contacted with one or more additional primers, each primer comprising a label and a first and a second region of nucleotides, wherein the first region of nucleotides is complementary to additional target nucleic acids.

9. The method of claim 1, wherein the second region of the primer has a sequence according to SEQ ID NO: 2 and the anti-primer has a sequence according to SEQ ID NO: 1.

10. A method comprising:
(a) contacting a sample to be tested for the relative amount of two target nucleic acids with
(i) a first primer comprising a first label, a first and a second region of nucleotides and a non-extendible linker between the first and second region of nucleotides, wherein the first region of nucleotides is complementary to a first target nucleic acid;
(ii) a second primer comprising a second label, a first and second region of nucleotides, and a non-extendible linker between the first and second region of nucleotides, wherein the first region of nucleotides is complementary to a second target nucleic acid and the second region of the second primer is complementary to the second region of the first primer and the second label is capable of quenching a detectable signal from the first label;
(iii) a first anti-primer comprising a third label and a nucleotide sequence complementary to the first primer, wherein the third label is capable of quenching a detectable signal from the first label; and
(iv) a second anti-primer comprising a fourth label and a nucleotide sequence complementary to the second primer, wherein the fourth label is capable of quenching a detectable signal from the second label, and
further wherein the sample is contacted under conditions wherein the primers specifically hybridize to the target nucleic acids, if present in the sample;
(b) performing an amplification reaction with the primers to produce amplification products having incorporated primers;
(c) detecting the relative amount of the two target nucleic acids in the sample by detecting the first label and the second label of the incorporated primers under conditions where the anti-primers specifically hybridize to the primers and hybridization between unincorporated primers and the anti-primers quenches the detectable signal from the unincorporated primers, and the second region of the first primer hybridizes to the second region of the second primer and hybridization between the second region of the first primer and the second region of the second primer quenches the detectable signal from the hybridized amplification products.

11. The method of claim 10, wherein the first label and second label each comprise a fluorophore.

12. The method of claim 11, wherein the fluorophores of the first and second labels are the same.

13. The method of claim 11, wherein the fluorophores of the first and second labels are different.

14. The method of claim 11, wherein the fluorophores of the first and second labels are independently selected from the group consisting of: FAM, TAMRA, ROX, CY5, CY3, and BODIPY.

15. The method of claim 10, wherein the third and fourth label each comprise a dark quencher.

16. The method of claim 10, wherein the melting temperatures of the first regions of the first and second primers are both higher than the melting temperatures of the anti-primers.

17. The method of claim 10, wherein the melting temperatures first regions of the first and second primers are from 5 to 10 degrees Celsius higher than the melting temperatures of the antiprimers.

18. The method of claim 10, wherein first and second target nucleic acids are alleles of a genetic locus.

19. The method of claim 18, wherein the first and second target nucleic acids differ by a single nucleotide.

20. The method of claim 10 comprising following step (c), determining the amount of each of the target nucleic acids by increasing the temperature to separate the hybridized second region of the first primer and the second region of the second primer and detecting the signal from one or both of the first label and the second label.


SEQUENCE LISTING (17 sequences)

SEQ ID NO: 1 - DNA, 17 bp, Artificial Sequence
  Description of Artificial Sequence Synthetic primer
ttccctcggatagcact

SEQ ID NO: 2 - DNA, 17 bp, Artificial Sequence
  Description of Artificial Sequence Synthetic primer
agtgctatccgagggaa

SEQ ID NO: 3 - DNA, 19 bp, Artificial Sequence
  Description of Artificial Sequence Synthetic primer
ggatgtgcggctcgtacac

SEQ ID NO: 4 - DNA, 39 bp, Artificial Sequence
  Description of Artificial Sequence Synthetic primer
agtgctatccgagggaatgacatggttgggactcttgac

SEQ ID NO: 5 - DNA, 37 bp, Artificial Sequence
  Description of Artificial Sequence Synthetic primer
gtgctatccgagggaacctgacctgccgtctagaaaa

SEQ ID NO: 6 - DNA, 18 bp, Artificial Sequence
  Description of Artificial Sequence Synthetic primer
ctccgacgcctgcttcac

SEQ ID NO: 7 - DNA, 36 bp, Artificial Sequence
  Description of Artificial Sequence Synthetic primer
agtgctatccgagggaagacagccccggatgagaac

SEQ ID NO: 8 - DNA, 22 bp, Artificial Sequence
  Description of Artificial Sequence Synthetic primer
aagaattgcaacagctcgattg

SEQ ID NO: 9 - DNA, 36 bp, Artificial Sequence
  Description of Artificial Sequence Synthetic primer
agtgctatccgagggaaccccagttgccgtctagga

SEQ ID NO: 10 - DNA, 23 bp, Artificial Sequence
  Description of Artificial Sequence Synthetic primer
cggacatactctgtttggcactt

SEQ ID NO: 11 - DNA, 39 bp, Artificial Sequence
  Description of Artificial Sequence Synthetic primer
agtgctatccgagggaagggcattatttgtgcactgaga

SEQ ID NO: 12 - DNA, 27 bp, Artificial Sequence
  Description of Artificial Sequence Synthetic primer
agcagcacggtatgagcaactgtcaga

SEQ ID NO: 13 - DNA, 38 bp, Artificial Sequence
  Description of Artificial Sequence Synthetic primer
agtgctatccgagggaatcctccttatgcctctatcat

SEQ ID NO: 14 - DNA, 21 bp, Artificial Sequence
  Description of Artificial Sequence Synthetic primer
ccgcgctttgatcaagagtcc

SEQ ID NO: 15 - DNA, 35 bp, Artificial Sequence
  Description of Artificial Sequence Synthetic primer
agtgctatccgagggaagaagaccagccagtgcac

SEQ ID NO: 16 - DNA, 36 bp, Artificial Sequence
  Description of Artificial Sequence Synthetic primer
agtgctatccgagggaatgaagaccagccagtgcat

SEQ ID NO: 17 - DNA, 21 bp, Artificial Sequence
  Description of Artificial Sequence Synthetic primer
caaggctttgccctcagggtt